(19) United States
(12) Patent Application Publication
(10) Pub. No.: US 2020/0347036 A1
(43) Pub. Date: Nov. 5, 2020

(54) SOLID FORMS OF AN HIV PROTEASE INHIBITOR

(71) Applicant: Gilead Sciences, Inc., Foster City, CA (US)

(72) Inventor: Bing Shi, Redwood City, CA (US)

(21) Appl. No.: 16/849,201

(22) Filed: Apr. 15, 2020

Related U.S. Application Data
(60) Provisional application No. 62/835,239, filed on Apr. 17, 2019.

(51) Int. Cl. C07D 403/14

(52) U.S. Cl. CPC C07D 403/14; A61K 45/06; C07B 2200/13

(57) ABSTRACT

The present disclosure relates to pharmaceutically acceptable salts and crystalline forms thereof, of a compound which is (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl (1- (difluoromethyl)cyclopropyl)carbamate, which is useful in the treatment of a Retroviridae viral infection including an infection caused by the HIV virus.

### CROSS-REFERENCE TO RELATED APPLICATIONS

This Application claims the benefit of U.S. Provisional Application 62/835,239 filed on Apr. 17, 2019. The entire contents of these applications are incorporated herein by reference in their entirety.

### TECHNICAL FIELD

The present disclosure relates to solid forms of an HIV protease inhibitor, including pharmaceutically acceptable salts and cocrystals of the inhibitor, as well as crystalline forms of the salts for use in the treatment of a Retroviridae viral infection including an infection caused by the HIV virus. The present disclosure also relates to pharmaceutical compositions containing the novel salts and crystalline forms thereof, and methods of treating or preventing a Retroviridae viral infection.

### BACKGROUND

Positive-single stranded RNA viruses comprising the Retroviridae family include those of the subfamily Orthoretrovirinae and genera Alpharetrovirus, Betaretrovirus, Gammaretrovirus, Deltaretrovirus, Epsilonretrovirus, Lentivirus, and Spumavirus which cause many human and animal diseases. Among the Lentivirus, HIV-1 infection in humans leads to depletion of T helper cells and immune dysfunction, producing immunodeficiency and vulnerability to opportunistic infections. Treating HIV-1 infections with highly active antiretroviral therapies (HAART) has proven to be effective at reducing viral load and significantly delaying disease progression (Hammer, S. M., et al.; JAMA 2008, 300: 555-570). However, these treatments could lead to the emergence of HIV strains that are resistant to current therapies (Taiwo, B., International Journal of Infectious Diseases 2009, 13:552-559; Smith, R. J., et al., Science 2010, 327:697-701). Therefore, there is an ongoing need to discover new antiretroviral agents and to develop methods for their preparation and purification as well as prepare improved pharmaceutical formulations of the same. The solid forms of the HIV protease inhibitor disclosed herein help meet these and other needs.

### SUMMARY

The present application provides solid forms of Compound 1:

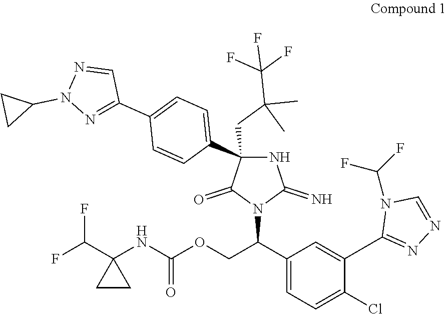 [SMILES: CC(C)(C[C@]1(c2ccc(-c3cnn(C4CC4)n3)cc2)NC(=N)N([C@H](COC(=O)NC2(C(F)F)CC2)c2ccc(Cl)c(-c3nncn3C(F)F)c2)C1=O)C(F)(F)F]

which include salts of Compound 1, as well as amorphous forms and crystalline forms of the salt.

The present application further provides solvates of the amorphous forms and solvates of the crystalline forms of the salts of Compound 1 disclosed herein.

The present application further provides pharmaceutical compositions comprising a pharmaceutically acceptable salt, amorphous form or crystalline form of Compound 1 disclosed herein, and at least one pharmaceutically acceptable excipient.

The present disclosure further provides amorphous forms of the pharmaceutically acceptable salts of Compound 1.

The present application further provides crystalline forms of the pharmaceutically acceptable salt of Compound 1.

The present application further provides methods of treating or preventing a human immunodeficiency virus (HIV) infection comprising administering a therapeutically effective amount of a pharmaceutically acceptable salt, an amorphous form or a crystalline form of the invention to a subject in need thereof.

The present application further provides the pharmaceutically acceptable salts, amorphous forms, and crystalline forms of the present invention for use in therapy.

The present application further provides the pharmaceutically acceptable salts, amorphous forms, or crystalline forms of the present invention for use in a method of treating or preventing a human immunodeficiency virus (HIV) infection, comprising administering a therapeutically effective amount of the salt, amorphous form or crystalline form to a subject in need thereof.

### BRIEF DESCRIPTION OF THE DRAWINGS

FIG. 1 shows an X-ray powder diffraction (XRPD) pattern characteristic of the THF solvate form of the hydrochloride salt of Compound 1.

FIG. 2 shows X-ray powder diffraction (XRPD) patterns characteristic of the hydrochloride salt of Compound 1, crystalline Form I, after desolvation of (a) a CPME solvate, (b) a THF/MTBE co-solvate, (c) a 2-BuOH solvate, (d) a 2-MeTHF solvate, or (e) an IPA solvate.

FIG. 3 shows an X-ray powder diffraction (XRPD) pattern characteristic of the hydrochloride salt of Compound 1, crystalline Form I.

FIG. 4 shows a thermogravimetric analysis (TGA) curve characteristic of the hydrochloride salt of Compound 1, crystalline Form I.

FIG. 5 shows a differential scanning calorimetry (DSC) curve characteristic of the hydrochloride salt of Compound 1, crystalline Form I.

FIG. 6 shows a dynamic vapor sorption (DVS) isotherm characteristic of the hydrochloride salt of Compound 1, crystalline Form I.

FIG. 7 shows an X-ray powder diffraction (XRPD) pattern characteristic of the DCM solvate form of the hydrochloride salt of Compound 1.

FIG. 8 shows an X-ray powder diffraction (XRPD) pattern characteristic of the hydrochloride salt of Compound 1, crystalline Form II.

FIG. 9 shows a thermogravimetric analysis (TGA) curve characteristic of the hydrochloride salt of Compound 1, crystalline Form II.

FIG. 10 shows a differential scanning calorimetry (DSC) curve characteristic of the hydrochloride salt of Compound 1, crystalline Form II.

FIG. 11 shows a dynamic vapor sorption (DVS) isotherm characteristic of the hydrochloride salt of Compound 1, crystalline Form II.

FIG. 12 shows an X-ray powder diffraction (XRPD) pattern characteristic of the EtOAc solvate form of the hydrochloride salt of Compound 1.

FIG. 13 shows a single crystal X-ray diffraction (SCXRD) molecular representation of the EtOAc solvate form of the hydrochloride salt of Compound 1.

FIG. 14 shows an X-ray powder diffraction (XRPD) pattern characteristic of the hydrochloride salt of Compound 1, crystalline Form III.

FIG. 15 shows a thermogravimetric analysis (TGA) curve characteristic of the hydrochloride salt of Compound 1, crystalline Form III.

FIG. 16 shows a differential scanning calorimetry (DSC) curve characteristic of the hydrochloride salt of Compound 1, crystalline Form III.

FIG. 17 shows a dynamic vapor sorption (DVS) isotherm characteristic of the hydrochloride salt of Compound 1, crystalline Form III.

FIG. 18 shows an X-ray powder diffraction (XRPD) pattern characteristic of Compound 1 hydrochloride salt, Material A.

FIG. 19 shows an X-ray powder diffraction (XRPD) pattern characteristic of Compound 1 hydrochloride salt, Material B.

FIG. 20 shows an X-ray powder diffraction (XRPD) pattern characteristic of the phosphate salt of Compound 1, crystalline Form I.

FIG. 21 shows a thermogravimetric analysis (TGA) curve characteristic of the phosphate salt of Compound 1, crystalline Form I.

FIG. 22 shows a differential scanning calorimetry (DSC) curve characteristic of the phosphate salt of Compound 1, crystalline Form I.

FIG. 23 shows a dynamic vapor sorption (DVS) isotherm characteristic of the phosphate salt of Compound 1, crystalline Form I.

FIG. 24 shows an X-ray powder diffraction (XRPD) pattern characteristic of the maleate salt of Compound 1, crystalline Form I.

FIG. 25 shows a single crystal X-ray diffraction (SCXRD) molecular representation of the maleate salt of Compound I, crystalline Form I.

FIG. 26 shows a thermogravimetric analysis (TGA) curve characteristic of the maleate salt of Compound 1, crystalline Form I.

FIG. 27 shows a differential scanning calorimetry (DSC) curve characteristic of the maleate salt of Compound 1, crystalline Form I.

FIG. 28 shows a dynamic vapor sorption (DVS) isotherm characteristic of the maleate salt of Compound 1, crystalline Form I.

FIG. 29 shows an X-ray powder diffraction (XRPD) pattern characteristic of the succinate salt of Compound 1, crystalline Form I.

FIG. 30 shows a single crystal X-ray diffraction (SCXRD) molecular representation of the succinate salt of Compound I, crystalline Form I.

FIG. 31 shows a thermogravimetric analysis (TGA) curve characteristic of the succinate salt of Compound 1, crystalline Form I.

FIG. 32 shows a differential scanning calorimetry (DSC) curve characteristic of the succinate salt of Compound 1, crystalline Form I.

FIG. 33 shows a dynamic vapor sorption (DVS) isotherm characteristic of the succinate salt of Compound 1, crystalline Form I.

FIG. 34 shows an X-ray powder diffraction (XRPD) pattern characteristic of the hemisuccinate salt of Compound 1, crystalline Form I.

FIG. 35 shows a thermogravimetric analysis (TGA) curve characteristic of the hemisuccinate salt of Compound 1, crystalline Form I.

FIG. 36 shows a differential scanning calorimetry (DSC) curve characteristic of the hemisuccinate salt of Compound 1, crystalline Form I.

FIG. 37 shows an X-ray powder diffraction (XRPD) pattern characteristic of the MIBK solvate form of Compound 2.

FIG. 38 shows an X-ray powder diffraction (XRPD) pattern characteristic of the MEK solvate form of Compound 2.

### DETAILED DESCRIPTION

The present invention relates to new solid forms of the HIV protease inhibitor (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl(1-(difluoromethyl)cyclopropyl)carbamate (i.e., Compound 1, see below). One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC). Accordingly, the compound structure for Compound 1 provided above may also be named or identified as provided by the chemical naming program ChemBioDraw Ultra 14.0: (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl(1-(difluoromethyl)cyclopropyl)carbamate.

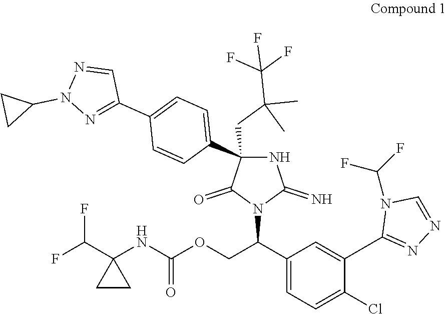 [SMILES: CC(C)(C[C@]1(c2ccc(-c3cnn(C4CC4)n3)cc2)NC(=N)N([C@H](COC(=O)NC2(C(F)F)CC2)c2ccc(Cl)c(-c3nncn3C(F)F)c2)C1=O)C(F)(F)F]

The solid forms of the invention include amorphous and crystalline salt forms of Compound 1. As used herein, “solid form” generally refers to a solid chemical substance that can be amorphous or crystalline. In some embodiments, the solid form of the invention is a salt of Compound 1 which can be amorphous or crystalline. Crystalline salts of Compound 1 can exist in different crystalline forms (i.e., have different polymorphic or pseudopolymorphic forms).

As used herein, “crystalline form” is meant to refer to a certain lattice configuration of a crystalline substance (e.g., a salt). Different crystalline forms of the same substance typically have different crystalline lattices (e.g., unit cells) which are attributed to different physical properties that are characteristic of each of the crystalline forms. In some instances, different lattice configurations have different water or solvent content.

According to the present invention, a crystalline form of a salt of Compound 1 can be useful in the synthesis and/or purification of Compound 1. For example, a crystalline form of a salt of Compound 1 can be an intermediate in the synthesis of Compound 1. In addition, different crystalline forms of salts of Compound 1 may have different properties with respect to bioavailability, stability, purity, and/or manufacturability for medical or pharmaceutical uses. Variations in the crystal structure of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, the crystalline forms of the salts of Compound 1 may provide advantages such as improving the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different crystalline forms of the salts of Compound 1 which may exhibit one or more of the favorable characteristics described above. The processes for the preparation of the crystalline forms described herein and characterization of these crystalline forms are described in detail below.

In some embodiments, the pharmaceutically acceptable salts described herein, or crystalline forms thereof, are purified or substantially isolated. By “substantially isolated” is meant that the salt or crystalline form thereof is at least partially or substantially separated from the environment in which it was formed or detected. Partial separation can include, for example, a composition enriched in the salt or crystalline form of the invention. Substantial separation can include compositions containing at least about 50%, at least about 60%, at least about 70%, at least about 80%, at least about 90%, at least about 95%, at least about 97%, or at least about 99% by weight of the salt or crystalline form of the invention. In some embodiments, the salt or crystalline form of the invention can be prepared with a purity of about 75% or more, 80% or more, 85% or more, 90% or more, 95% or more, 98% or more, or 99% or more.

The different crystalline forms can be identified by solid state characterization methods such as by X-ray powder diffraction (XRPD) or single crystal X-ray diffraction (SCXRD). Other characterization methods such as differential scanning calorimetry (DSC), thermogravimetric (TGA) analysis, or dynamic vapor sorption (DVS) analysis further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations. The XRPD patterns provided herein were obtained using CuK_{α1} radiation.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

The present invention provides crystalline forms of certain compounds or salts thereof. In some embodiments, the crystalline form may be substantially anhydrous. In some embodiments, the crystalline form may be hydrated or solvated.

### Compound 1 Hydrochloride Salt

In some embodiments, Compound 1 can be isolated as a hydrochloride salt which can be amorphous or crystalline. In some embodiments, the hydrochloride salt of Compound 1 is crystalline.

In some embodiments, the crystalline hydrochloride salt of Compound 1 is selected from crystalline Form I, crystalline Form II, and crystalline Form III.

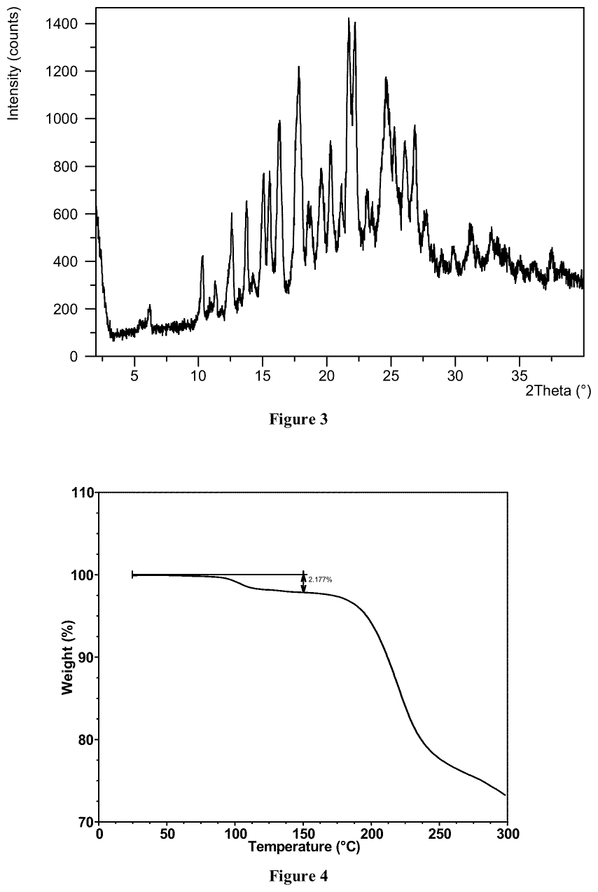

In some embodiments, crystalline Form I of the hydrochloride salt of Compound 1 is characterized by an XRPD pattern substantially as shown in FIG. 3.

In some embodiments, crystalline Form I of the hydrochloride salt of Compound 1 is characterized by an XRPD pattern comprising one peak, in terms of 2-theta±0.2°, selected from 6.2°, 10.4°, 12.6°, 13.7°, 16.3°, 17.8°, 21.7°, 22.2°, and 26.8°.

In some embodiments, crystalline Form I of the hydrochloride salt of Compound 1 is characterized by an XRPD pattern comprising two peaks, in terms of 2-theta±0.2°, selected from 6.2°, 10.4°, 12.6°, 13.7°, 16.3°, 17.8°, 21.7°, 22.2°, and 26.8°.

In some embodiments, crystalline Form I of the hydrochloride salt of Compound 1 is characterized by an XRPD pattern comprising three peaks, in terms of 2-theta±0.2°, selected from 6.2°, 10.4°, 12.6°, 13.7°, 16.3°, 17.8°, 21.7°, 22.2°, and 26.8°.

In some embodiments, crystalline Form I of the hydrochloride salt of Compound 1 is characterized by an XRPD pattern comprising four peaks, in terms of 2-theta±0.2°, selected from 6.2°, 10.4°, 12.6°, 13.7°, 16.3°, 17.8°, 21.7°, 22.2°, and 26.8°.

In some embodiments, crystalline Form I of the hydrochloride salt of Compound 1 is characterized by an XRPD pattern comprising five peaks, in terms of 2-theta±0.2°, selected from 6.2°, 10.4°, 12.6°, 13.7°, 16.3°, 17.8°, 21.7°, 22.2°, and 26.8°.

In some embodiments, crystalline Form I of the hydrochloride salt of Compound 1 is characterized by an XRPD pattern comprising six peaks, in terms of 2-theta±0.2°, selected from 6.2°, 10.4°, 12.6°, 13.7°, 16.3°, 17.8°, 21.7°, 22.2°, and 26.8°.

In some embodiments, crystalline Form I of the hydrochloride salt of Compound 1 is characterized by an XRPD pattern comprising seven peaks, in terms of 2-theta±0.2°, selected from 6.2°, 10.4°, 12.6°, 13.7°, 16.3°, 17.8°, 21.7°, 22.2°, and 26.8°.

In some embodiments, crystalline Form I of the hydrochloride salt of Compound 1 is characterized by an XRPD pattern comprising eight peaks, in terms of 2-theta±0.2°, selected from 6.2°, 10.4°, 12.6°, 13.7°, 16.3°, 17.8°, 21.7°, 22.2°, and 26.8°.

In some embodiments, crystalline Form I of the hydrochloride salt of Compound 1 is characterized by an XRPD pattern comprising peaks at 10.4, 12.6, and 22.2 degrees 2θ (±0.2 degrees 2θ).

In some embodiments, crystalline Form I of the hydrochloride salt of Compound 1 is characterized by an XRPD pattern comprising peaks at 6.2, 10.4, 12.6, and 22.2 degrees 2θ (±0.2 degrees 2θ).

In some embodiments, crystalline Form I of the hydrochloride salt of Compound 1 is characterized by an XRPD pattern comprising peaks at 6.2, 10.4, 12.6, 17.8, and 22.2 degrees 2θ (±0.2 degrees 2θ).

In some embodiments, crystalline Form I of the hydrochloride salt of Compound 1 is characterized by an XRPD pattern comprising peaks at 6.2, 10.4, 12.6, 17.8, 21.7, and 22.2 degrees 2θ (±0.2 degrees 2θ).

In some embodiments, crystalline Form I of the hydrochloride salt of Compound 1 is characterized by an XRPD pattern comprising peaks at 6.2, 10.4, 12.6, 13.7, 17.8, 21.7, and 22.2.

In some embodiments, crystalline Form I of the hydrochloride salt of Compound 1 is characterized by an XRPD pattern comprising peaks at 6.2, 10.4, 12.6, 13.7, 16.3, 17.8, 21.7, and 22.2 degrees 2θ (±0.2 degrees 2θ).

In some embodiments, crystalline Form I of the hydrochloride salt of Compound 1 is characterized by an XRPD pattern comprising peaks at 6.2, 10.4, 12.6, 13.7, 16.3, 17.8, 21.7, 22.2, and 26.8 degrees 2θ (±0.2 degrees 2θ).

In some embodiments, crystalline Form I of the hydrochloride salt of Compound 1 is characterized by a TGA thermogram substantially as shown in FIG. 4.

In some embodiments, crystalline Form I of the hydrochloride salt of Compound 1 is characterized by weight loss of about 2% from 25° C. to 150° C. as determined by TGA.

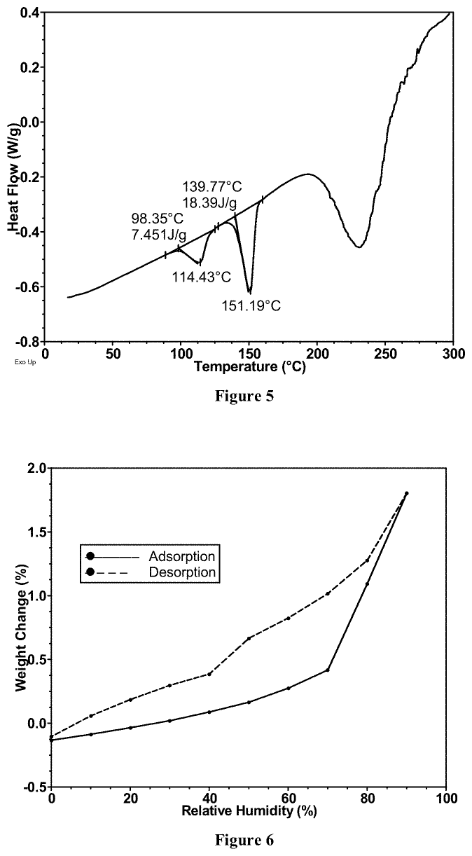

In some embodiments, crystalline Form I of the hydrochloride salt of Compound 1 is characterized by a DSC thermogram substantially as shown in FIG. 5.

In some embodiments, crystalline Form I of the hydrochloride salt of Compound 1 is characterized by a DSC thermogram having a melting onset of about 140° C.

In some embodiments, crystalline Form I of the hydrochloride salt of Compound 1 is characterized by a DVS sorption isotherm substantially as shown in FIG. 6.

In some embodiments, crystalline Form I of the hydrochloride salt of Compound 1 is characterized by about 1.8% water uptake over a range of 10% to 90% RH at 25° C. as determined by DVS.

In some embodiments, crystalline Form II of the hydrochloride salt of Compound 1 is characterized by an XRPD pattern substantially as shown in FIG. 8.

In some embodiments, crystalline Form II of the hydrochloride salt of Compound 1 is characterized by an XRPD pattern comprising one peak, in terms of 2-theta±0.2°, selected from 6.5°, 7.2°, 8.5°, 11.3°, 13.4°, 14.3°, 15.7°, 17.0°, and 17.7°.

In some embodiments, crystalline Form II of the hydrochloride salt of Compound 1 is characterized by an XRPD pattern comprising two peaks, in terms of 2-theta±0.2°, selected from 6.5°, 7.2°, 8.5°, 11.3°, 13.4°, 14.3°, 15.7°, 17.0°, and 17.7°.

In some embodiments, crystalline Form II of the hydrochloride salt of Compound 1 is characterized by an XRPD pattern comprising three peaks, in terms of 2-theta±0.2°, selected from 6.5°, 7.2°, 8.5°, 11.3°, 13.4°, 14.3°, 15.7°, 17.0°, and 17.7°.

In some embodiments, crystalline Form II of the hydrochloride salt of Compound 1 is characterized by an XRPD pattern comprising four peaks, in terms of 2-theta±0.2°, selected from 6.5°, 7.2°, 8.5°, 11.3°, 13.4°, 14.3°, 15.7°, 17.0°, and 17.7°.

In some embodiments, crystalline Form II of the hydrochloride salt of Compound 1 is characterized by an XRPD pattern comprising five peaks, in terms of 2-theta±0.2°, selected from 6.5°, 7.2°, 8.5°, 11.3°, 13.4°, 14.3°, 15.7°, 17.0°, and 17.7°.

In some embodiments, crystalline Form II of the hydrochloride salt of Compound 1 is characterized by an XRPD pattern comprising six peaks, in terms of 2-theta±0.2°, selected from 6.5°, 7.2°, 8.5°, 11.3°, 13.4°, 14.3°, 15.7°, 17.0°, and 17.7°.

In some embodiments, crystalline Form II of the hydrochloride salt of Compound 1 is characterized by an XRPD pattern comprising seven peaks, in terms of 2-theta±0.2°, selected from 6.5°, 7.2°, 8.5°, 11.3°, 13.4°, 14.3°, 15.7°, 17.0°, and 17.7°.

In some embodiments, crystalline Form II of the hydrochloride salt of Compound 1 is characterized by an XRPD pattern comprising eight peaks, in terms of 2-theta±0.2°, selected from 6.5°, 7.2°, 8.5°, 11.3°, 13.4°, 14.3°, 15.7°, 17.0°, and 17.7°.

In some embodiments, crystalline Form II of the hydrochloride salt of Compound 1 is characterized by an XRPD pattern comprising peaks at 6.5, 7.2, and 14.3 degrees 2θ (±0.2 degrees 2θ).

In some embodiments, crystalline Form II of the hydrochloride salt of Compound 1 is characterized by an XRPD pattern comprising peaks at 6.5, 7.2, 14.3, and 15.7 degrees 2θ (±0.2 degrees 2θ).

In some embodiments, crystalline Form II of the hydrochloride salt of Compound 1 is characterized by an XRPD pattern comprising peaks at 6.5, 7.2, 14.3, 15.7, and 17.0 degrees 2θ (±0.2 degrees 2θ).

In some embodiments, crystalline Form II of the hydrochloride salt of Compound 1 is characterized by an XRPD pattern comprising peaks at 6.5, 7.2, 14.3, 15.7, 17.0, and 17.7 degrees 2θ (±0.2 degrees 2θ).

In some embodiments, crystalline Form II of the hydrochloride salt of Compound 1 is characterized by an XRPD pattern comprising peaks at 6.5, 7.2, 8.5, 14.3, 15.7, 17.0, and 17.7 degrees 2θ (±0.2 degrees 2θ).

In some embodiments, crystalline Form II of the hydrochloride salt of Compound 1 is characterized by an XRPD pattern comprising peaks at 6.5, 7.2, 8.5, 11.3, 14.3, 15.7, 17.0, and 17.7 degrees 2θ (±0.2 degrees 2θ).

In some embodiments, crystalline Form II of the hydrochloride salt of Compound 1 is characterized by an XRPD pattern comprising peaks at 6.5, 7.2, 8.5, 11.3, 13.4, 14.3, 15.7, 17.0, and 17.7 degrees 2θ (±0.2 degrees 2θ).

In some embodiments, crystalline Form II of the hydrochloride salt of Compound 1 is characterized by a TGA thermogram substantially as shown in FIG. 9.

In some embodiments, crystalline Form II of the hydrochloride salt of Compound 1 is characterized by weight loss of about 1% from 25° C. to 70° C. and by weight loss of about 0.4% from 70° C. to 150° C. as determined by TGA.

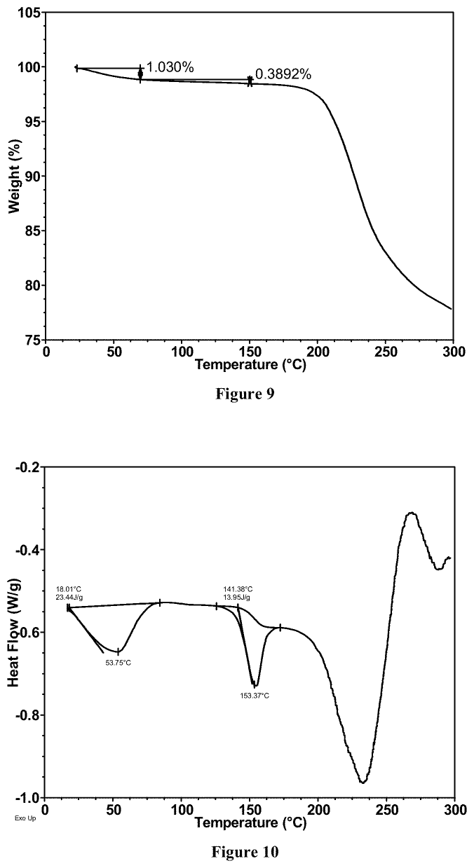

In some embodiments, crystalline Form II of the hydrochloride salt of Compound 1 is characterized by a DSC thermogram substantially as shown in FIG. 10.

In some embodiments, crystalline Form II of the hydrochloride salt of Compound 1 is characterized by a DSC thermogram having a melting onset of about 141° C.

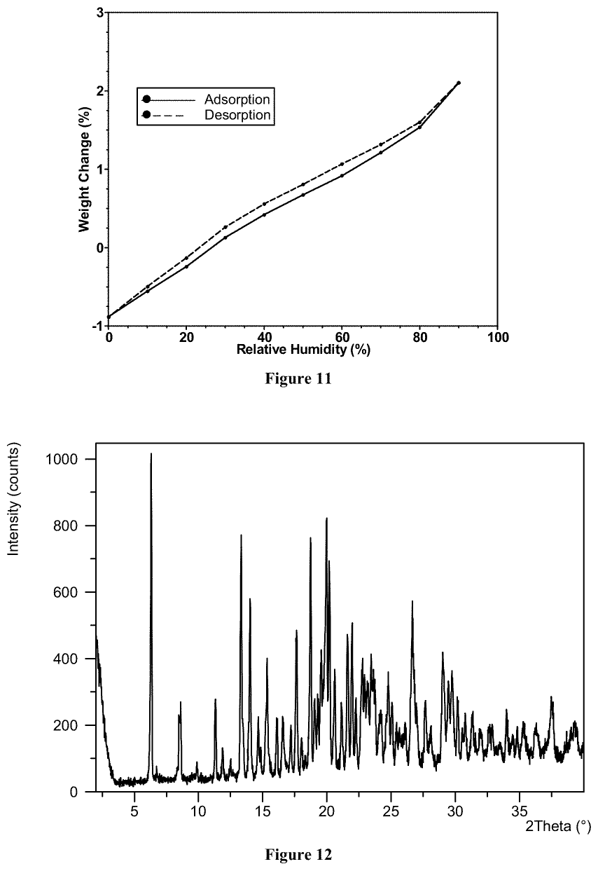

In some embodiments, crystalline Form II of the hydrochloride salt of Compound 1 is characterized by a DVS sorption isotherm substantially as shown in FIG. 11.

In some embodiments, crystalline Form II of the hydrochloride salt of Compound 1 is characterized by about 3.0% water uptake over a range of 0% to 90% RH at 25° C. as determined by DVS.

In some embodiments, crystalline Form III of the hydrochloride salt of Compound 1 is characterized by an XRPD pattern substantially as shown in FIG. 14.

In some embodiments, crystalline Form III of the hydrochloride salt of Compound 1 is characterized by an XRPD pattern comprising one peak, in terms of 2-theta±0.2°, selected from 6.3°, 6.6°, 10.9°, 13.7°, 15.3°, 17.3°, 18.5°, 20.0°, and 25.1°.

In some embodiments, crystalline Form III of the hydrochloride salt of Compound 1 is characterized by an XRPD pattern comprising two peaks, in terms of 2-theta±0.2°, selected from 6.3°, 6.6°, 10.9°, 13.7°, 15.3°, 17.3°, 18.5°, 20.0°, and 25.1°.

In some embodiments, crystalline Form III of the hydrochloride salt of Compound 1 is characterized by an XRPD pattern comprising three peaks, in terms of 2-theta±0.2°, selected from 6.3°, 6.6°, 10.9°, 13.7°, 15.3°, 17.3°, 18.5°, 20.0°, and 25.1°.

In some embodiments, crystalline Form III of the hydrochloride salt of Compound 1 is characterized by an XRPD pattern comprising four peaks, in terms of 2-theta±0.2°, selected from 6.3°, 6.6°, 10.9°, 13.7°, 15.3°, 17.3°, 18.5°, 20.0°, and 25.1°.

In some embodiments, crystalline Form III of the hydrochloride salt of Compound 1 is characterized by an XRPD pattern comprising five peaks, in terms of 2-theta±0.2°, selected from 6.3°, 6.6°, 10.9°, 13.7°, 15.3°, 17.3°, 18.5°, 20.0°, and 25.1°.

In some embodiments, crystalline Form III of the hydrochloride salt of Compound 1 is characterized by an XRPD pattern comprising six peaks, in terms of 2-theta±0.2°, selected from 6.3°, 6.6°, 10.9°, 13.7°, 15.3°, 17.3°, 18.5°, 20.0°, and 25.1°.

In some embodiments, crystalline Form III of the hydrochloride salt of Compound 1 is characterized by an XRPD pattern comprising seven peaks, in terms of 2-theta±0.2°, selected from 6.3°, 6.6°, 10.9°, 13.7°, 15.3°, 17.3°, 18.5°, 20.0°, and 25.1°.

In some embodiments, crystalline Form III of the hydrochloride salt of Compound 1 is characterized by an XRPD pattern comprising eight peaks, in terms of 2-theta±0.2°, selected from 6.3°, 6.6°, 10.9°, 13.7°, 15.3°, 17.3°, 18.5°, 20.0°, and 25.1°.

In some embodiments, crystalline Form III of the hydrochloride salt of Compound 1 is characterized by an XRPD pattern comprising peaks at 10.9, 15.3, and 18.5 degrees 2θ (±0.2 degrees 2θ).

In some embodiments, crystalline Form III of the hydrochloride salt of Compound 1 is characterized by an XRPD pattern comprising peaks at 6.3, 10.9, 15.3, and 18.5 degrees 2θ (±0.2 degrees 2θ).

In some embodiments, crystalline Form III of the hydrochloride salt of Compound 1 is characterized by an XRPD pattern comprising peaks at 6.3 or 6.6 degrees, 10.9, 15.3, and 18.5 degrees 2θ (±0.2 degrees 2θ).

In some embodiments, crystalline Form III of the hydrochloride salt of Compound 1 is characterized by an XRPD pattern comprising peaks at 6.3, 6.6, 10.9, 15.3, and 18.5 degrees 2θ (±0.2 degrees 2θ).

In some embodiments, crystalline Form III of the hydrochloride salt of Compound 1 is characterized by an XRPD pattern comprising peaks at 6.3, 6.6, 10.9, 15.3, 18.5, and 20.0 degrees 2θ (±0.2 degrees 2θ).

In some embodiments, crystalline Form III of the hydrochloride salt of Compound 1 is characterized by an XRPD pattern comprising peaks at 6.3, 6.6, 10.9, 13.7, 15.3, 18.5, and 20.0 degrees 2θ (±0.2 degrees 2θ).

In some embodiments, crystalline Form III of the hydrochloride salt of Compound 1 is characterized by an XRPD pattern comprising peaks at 6.3, 6.6, 10.9, 13.7, 15.3, 17.3, 18.5, and 20.0 degrees 2θ (±0.2 degrees 2θ).

In some embodiments, crystalline Form III of the hydrochloride salt of Compound 1 is characterized by an XRPD pattern comprising peaks at 6.3, 6.6, 10.9, 13.7, 15.3, 17.3, 18.5, 20.0, and 25.1° degrees 2θ (±0.2 degrees 2θ).

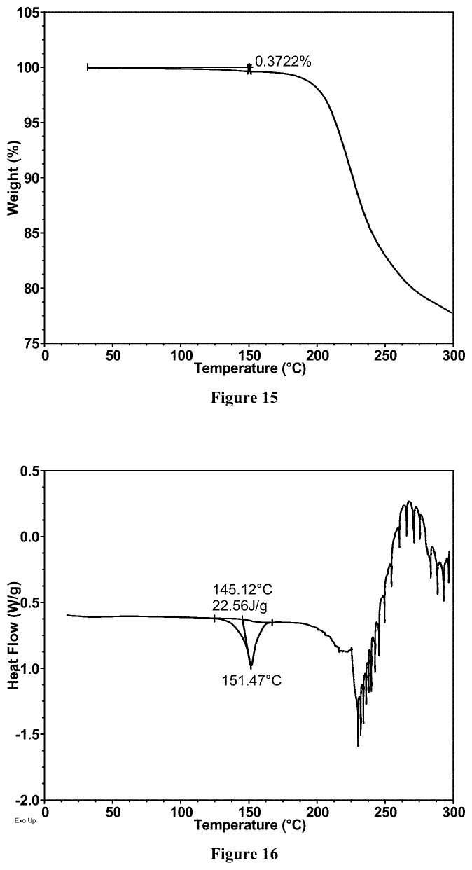

In some embodiments, crystalline Form III of the hydrochloride salt of Compound 1 is characterized by a TGA thermogram substantially as shown in FIG. 15.

In some embodiments, crystalline Form III of the hydrochloride salt of Compound 1 is characterized by weight loss of about 0.4% from 25° C. to 150° C. as determined by TGA.

In some embodiments, crystalline Form III of the hydrochloride salt of Compound 1 is characterized by a DSC thermogram substantially as shown in FIG. 16.

In some embodiments, crystalline Form III of the hydrochloride salt of Compound 1 is characterized by a DSC thermogram having a melting onset of about 145° C.

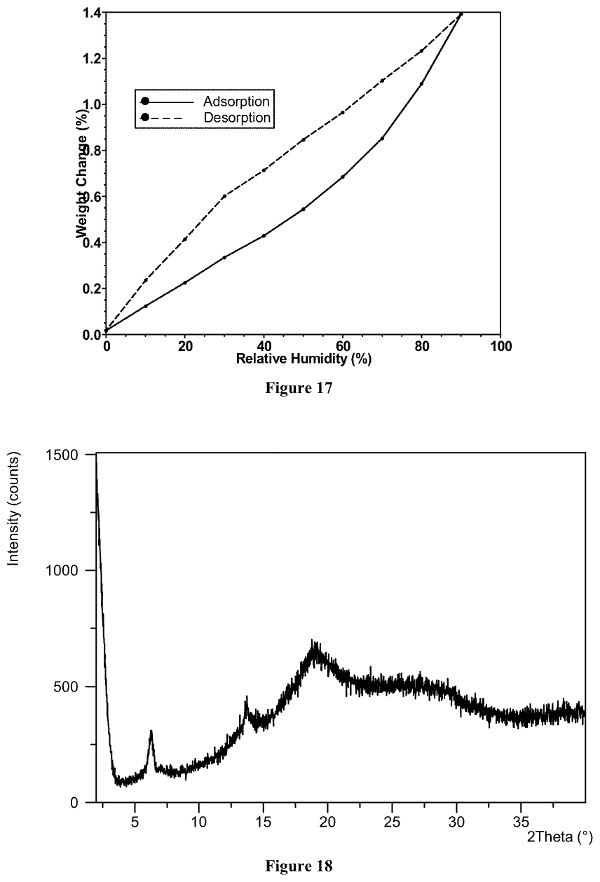

In some embodiments, crystalline Form III of the hydrochloride salt of Compound 1 is characterized by a DVS sorption isotherm substantially as shown in FIG. 17.

In some embodiments, crystalline Form III of the hydrochloride salt of Compound 1 is characterized by about 1.4% water uptake over a range of 0% to 90% RH at 25° C. as determined by DVS.

In some embodiments, the crystalline hydrochloride salt of Compound 1 is unsolvated.

In some embodiments, the crystalline hydrochloride salt of Compound 1 is solvated.

In some embodiments, the crystalline hydrochloride salt of Compound 1 is a tetrahydrofuran solvate.

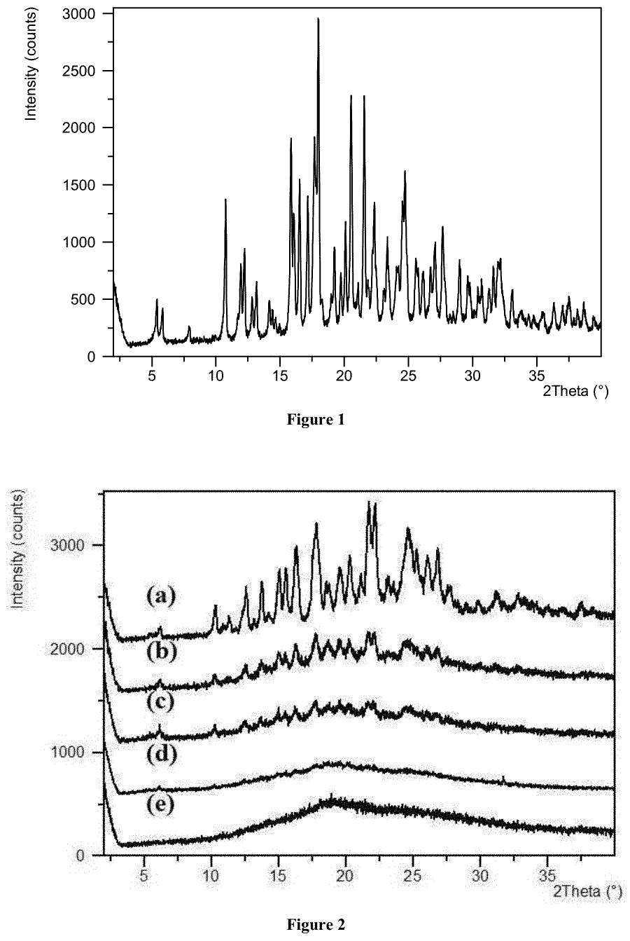

In some embodiments, the crystalline hydrochloride salt of Compound 1, tetrahydrofuran solvate has an XRPD profile substantially as shown in FIG. 1.

In some embodiments, the crystalline hydrochloride salt of Compound 1 is a dichloromethane solvate.

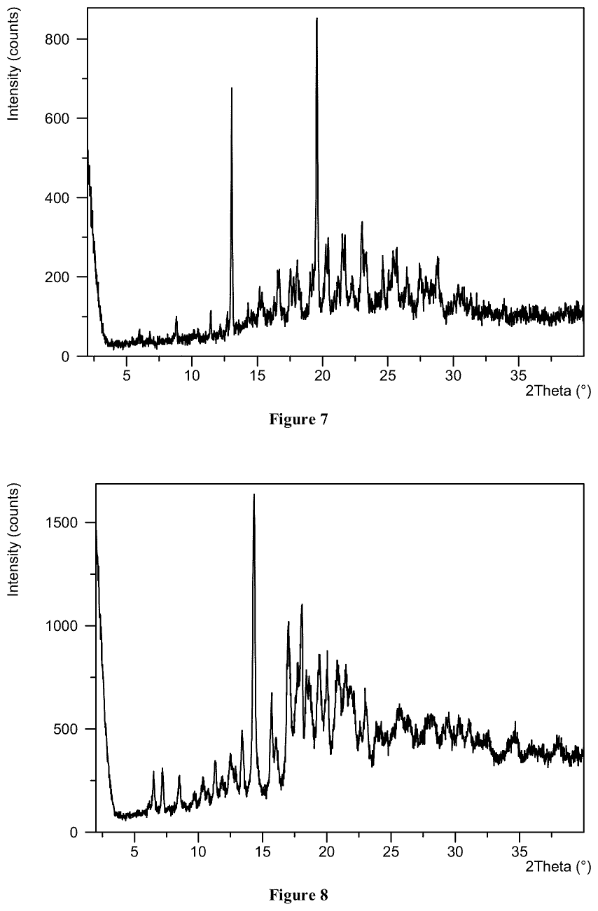

In some embodiments, the crystalline hydrochloride salt of Compound 1, dichloromethane solvate is characterized by an XRPD pattern substantially as shown in FIG. 7.

In some embodiments, the crystalline hydrochloride salt of Compound 1 is an ethyl acetate solvate.

In some embodiments, the crystalline hydrochloride salt of Compound 1, ethyl acetate solvate is characterized by an XRPD pattern substantially as shown in FIG. 12.

In some embodiments, the crystalline hydrochloride salt of Compound 1, ethyl acetate solvate has an SCXRD molecular representation substantially as shown in Table 4.

In some embodiments, the crystalline hydrochloride salt of Compound 1, ethyl acetate solvate is characterized by a unit cell as determined by single crystal X-ray crystallography (SCXRD) of the following dimensions: a=11.65967(19) Å; b=13.6291(3) Å; c=15.3692(3) Å; α=79.8278(19)°; β=68.0006(18)°; and γ=78.9173(17)°.

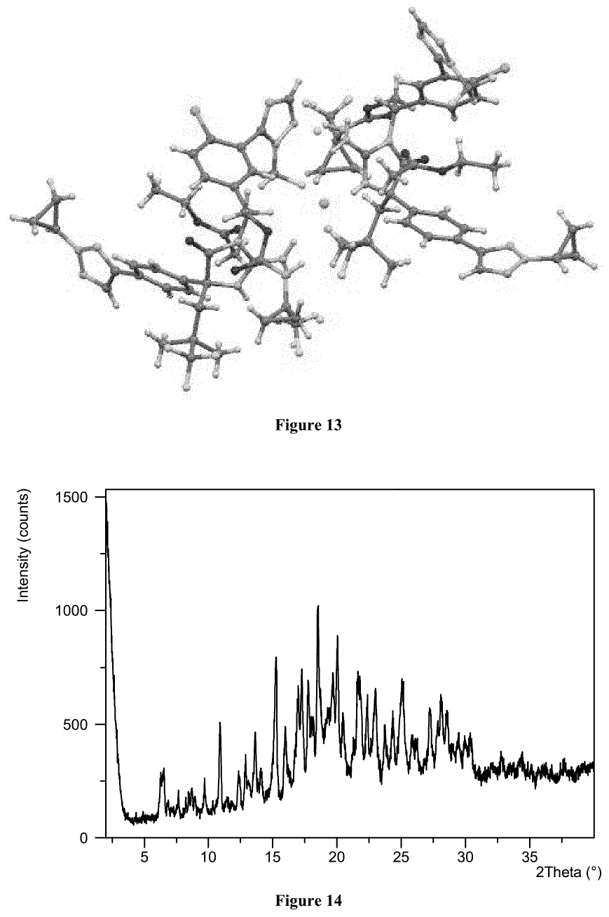

In some embodiments, the crystalline hydrochloride salt of Compound 1, ethyl acetate solvate has an SCXRD molecular representation substantially as shown in FIG. 13.

In some embodiments, the crystalline hydrochloride salt of Compound 1, ethyl acetate solvate has an SCXRD molecular representation substantially as shown in Table 4.

In some embodiments, Compound 1 hydrochloride can be isolated in the form of Material A. In some embodiments Material A of Compound 1 hydrochloride salt has an XRPD profile substantially as shown in FIG. 18.

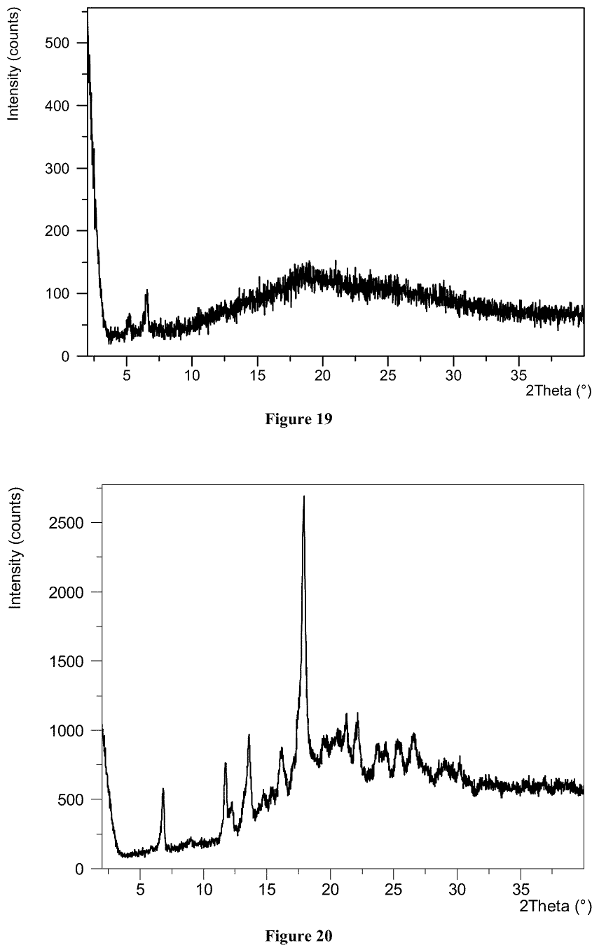

In some embodiments, Compound 1 hydrochloride can be isolated in the form of Material B. In some embodiments Material B of Compound 1 hydrochloride salt has an XRPD profile substantially as shown in FIG. 19.

### Compound 1 Phosphate Salt

In some embodiments, Compound 1 can be isolated as a phosphate salt which can be amorphous or crystalline. In some embodiments, the phosphate salt of Compound 1 is crystalline.

In some embodiments, the crystalline phosphate salt of Compound 1 is crystalline Form I.

In some embodiments, crystalline Form I of the phosphate salt of Compound 1 is characterized by an XRPD pattern substantially as shown in FIG. 20.

In some embodiments, crystalline Form I of the phosphate salt of Compound 1 is characterized by an XRPD pattern comprising one peak, in terms of 2-theta±0.2°, selected from 6.8°, 11.8°, 12.2°, 13.4°, 16.1°, 17.9°, 21.2°, 22.0°, and 25.3°.

In some embodiments, crystalline Form I of the phosphate salt of Compound 1 is characterized by an XRPD pattern comprising two peaks, in terms of 2-theta±0.2°, selected from 6.8°, 11.8°, 12.2°, 13.4°, 16.1°, 17.9°, 21.2°, 22.0°, and 25.3°.

In some embodiments, crystalline Form I of the phosphate salt of Compound 1 is characterized by an XRPD pattern comprising three peaks, in terms of 2-theta±0.2°, selected from 6.8°, 11.8°, 12.2°, 13.4°, 16.1°, 17.9°, 21.2°, 22.0°, and 25.3°.

In some embodiments, crystalline Form I of the phosphate salt of Compound 1 is characterized by an XRPD pattern comprising four peaks, in terms of 2-theta±0.2°, selected from 6.8°, 11.8°, 12.2°, 13.4°, 16.1°, 17.9°, 21.2°, 22.0°, and 25.3°.

In some embodiments, crystalline Form I of the phosphate salt of Compound 1 is characterized by an XRPD pattern comprising five peaks, in terms of 2-theta±0.2°, selected from 6.8°, 11.8°, 12.2°, 13.4°, 16.1°, 17.9°, 21.2°, 22.0°, and 25.3°.

In some embodiments, crystalline Form I of the phosphate salt of Compound 1 is characterized by an XRPD pattern comprising six peaks, in terms of 2-theta±0.2°, selected from 6.8°, 11.8°, 12.2°, 13.4°, 16.1°, 17.9°, 21.2°, 22.0°, and 25.3°.

In some embodiments, crystalline Form I of the phosphate salt of Compound 1 is characterized by an XRPD pattern comprising seven peaks, in terms of 2-theta±0.2°, selected from 6.8°, 11.8°, 12.2°, 13.4°, 16.1°, 17.9°, 21.2°, 22.0°, and 25.3°.

In some embodiments, crystalline Form I of the phosphate salt of Compound 1 is characterized by an XRPD pattern comprising eight peaks, in terms of 2-theta±0.2°, selected from 6.8°, 11.8°, 12.2°, 13.4°, 16.1°, 17.9°, 21.2°, 22.0°, and 25.3°.

In some embodiments, crystalline Form I of the phosphate salt of Compound 1 is characterized by an XRPD pattern comprising three peaks at 6.8, 11.8, and 17.9 degrees 2θ (±0.2 degrees 2θ).

In some embodiments, crystalline Form I of the phosphate salt of Compound 1 is characterized by an XRPD pattern comprising four peaks at 6.8, 11.8, 13.4, and 17.9 degrees 2θ (±0.2 degrees 2θ).

In some embodiments, crystalline Form I of the phosphate salt of Compound 1 is characterized by an XRPD pattern comprising five peaks at 6.8, 11.8, 13.4, 16.1, and 17.9 degrees 2θ (±0.2 degrees 2θ).

In some embodiments, crystalline Form I of the phosphate salt of Compound 1 is characterized by an XRPD pattern comprising six peaks at 6.8, 11.8, 13.4, 16.1, 17.9, and 21.2 degrees 2θ (±0.2 degrees 2θ).

In some embodiments, crystalline Form I of the phosphate salt of Compound 1 is characterized by an XRPD pattern comprising seven peaks at 6.8, 11.8, 12.2, 13.4, 16.1, 17.9, and 21.2 degrees 2θ (±0.2 degrees 2θ).

In some embodiments, crystalline Form I of the phosphate salt of Compound 1 is characterized by an XRPD pattern comprising eight peaks at 6.8, 11.8, 12.2, 13.4, 16.1, 17.9, 21.2, and 22.0 degrees 2θ (±0.2 degrees 2θ).

In some embodiments, crystalline Form I of the phosphate salt of Compound 1 is characterized by an XRPD pattern comprising nine peaks at 6.8, 11.8, 12.2, 13.4, 16.1, 17.9, 21.2, 22.0, and 25.3 degrees 2θ (±0.2 degrees 2θ).

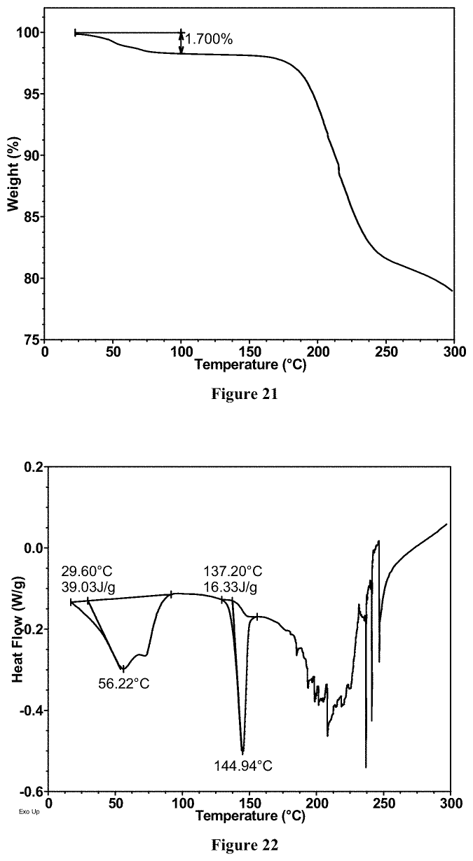

In some embodiments, crystalline Form I of the phosphate salt of Compound 1 is characterized by a TGA thermogram substantially as shown in FIG. 21.

In some embodiments, crystalline Form I of the phosphate salt of Compound 1 is characterized by weight loss of about 1.7% from 25° C. to 100° C. as determined by TGA.

In some embodiments, crystalline Form I of the phosphate salt of Compound 1 is characterized by a DSC thermogram substantially as shown in FIG. 22.

In some embodiments, crystalline Form I of the phosphate salt of Compound 1 is characterized by a DSC thermogram having a melting onset of about 137° C.

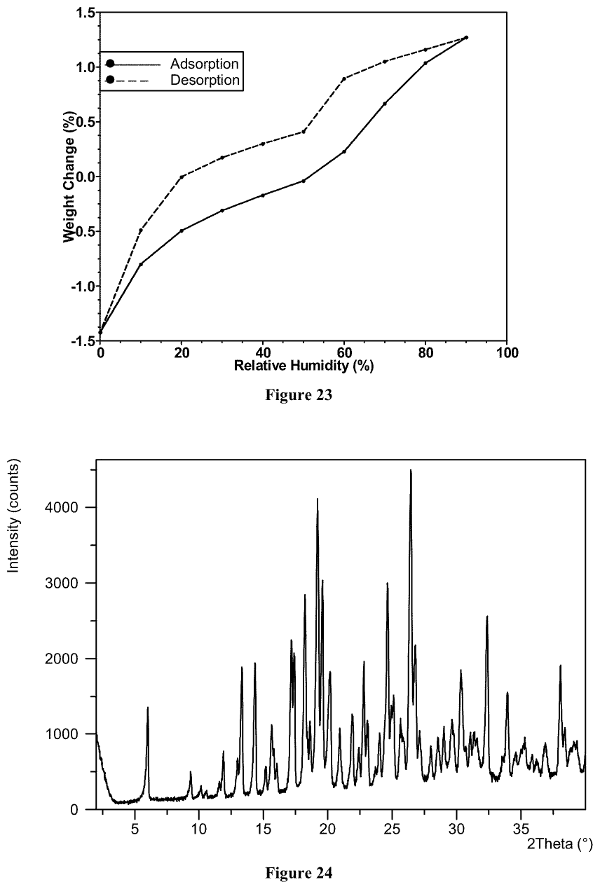

In some embodiments, crystalline Form I of the phosphate salt of Compound 1 is characterized by a DVS sorption isotherm substantially as shown in FIG. 23.

In some embodiments, crystalline Form I of the phosphate salt of Compound 1 is characterized by about 2.7% water uptake over a range of 10% to 90% RH at 25° C. as determined by DVS.

### Compound 1 Maleate Salt

In some embodiments, Compound 1 can be isolated as a maleate salt which can be amorphous or crystalline.

In some embodiments, the crystalline maleate salt or of Compound 1 is crystalline Form I.

In some embodiments, crystalline Form I of the maleate salt of Compound 1 has an XRPD pattern substantially as shown in FIG. 24.

In some embodiments, crystalline Form I of the maleate salt of Compound 1 is characterized by an XRPD pattern comprising one peak, in terms of 2-theta±0.2°, selected from 6.0°, 13.3°, 14.4°, 17.2°, 18.2°, 19.2°, 19.6°, 24.6°, and 26.5°.

In some embodiments, crystalline Form I of the maleate salt of Compound 1 is characterized by an XRPD pattern comprising two peaks, in terms of 2-theta±0.2°, selected from 6.0°, 13.3°, 14.4°, 17.2°, 18.2°, 19.2°, 19.6°, 24.6°, and 26.5°.

In some embodiments, crystalline Form I of the maleate salt of Compound 1 is characterized by an XRPD pattern comprising three peaks, in terms of 2-theta±0.2°, selected from 6.0°, 13.3°, 14.4°, 17.2°, 18.2°, 19.2°, 19.6°, 24.6°, and 26.5°.

In some embodiments, crystalline Form I of the maleate salt of Compound 1 is characterized by an XRPD pattern comprising four peaks, in terms of 2-theta±0.2°, selected from 6.0°, 13.3°, 14.4°, 17.2°, 18.2°, 19.2°, 19.6°, 24.6°, and 26.5°.

In some embodiments, crystalline Form I of the maleate salt of Compound 1 is characterized by an XRPD pattern comprising five peaks, in terms of 2-theta±0.2°, selected from 6.0°, 13.3°, 14.4°, 17.2°, 18.2°, 19.2°, 19.6°, 24.6°, and 26.5°.

In some embodiments, crystalline Form I of the maleate salt of Compound 1 is characterized by an XRPD pattern comprising six peaks, in terms of 2-theta±0.2°, selected from 6.0°, 13.3°, 14.4°, 17.2°, 18.2°, 19.2°, 19.6°, 24.6°, and 26.5°.

In some embodiments, crystalline Form I of the maleate salt of Compound 1 is characterized by an XRPD pattern comprising seven peaks, in terms of 2-theta±0.2°, selected from 6.0°, 13.3°, 14.4°, 17.2°, 18.2°, 19.2°, 19.6°, 24.6°, and 26.5°.

In some embodiments, crystalline Form I of the maleate salt of Compound 1 is characterized by an XRPD pattern comprising eight peaks, in terms of 2-theta±0.2°, selected from 6.0°, 13.3°, 14.4°, 17.2°, 18.2°, 19.2°, 19.6°, 24.6°, and 26.5°.

In some embodiments, crystalline Form I of the maleate salt of Compound 1 is characterized by an XRPD pattern comprising three peaks at 6.0, 19.2, and 26.5 degrees 2θ (±0.2 degrees 2θ).

In some embodiments, crystalline Form I of the maleate salt of Compound 1 is characterized by an XRPD pattern comprising four peaks at 6.0, 13.3, 19.2, and 26.5 degrees 2θ (±0.2 degrees 2θ).

In some embodiments, crystalline Form I of the maleate salt of Compound 1 is characterized by an XRPD pattern comprising five peaks at 6.0, 13.3, 14.4, 19.2, and 26.5 degrees 2θ (±0.2 degrees 2θ).

In some embodiments, crystalline Form I of the maleate salt of Compound 1 is characterized by an XRPD pattern comprising six peaks at 6.0, 13.3, 14.4, 18.2, 19.2, and 26.5 degrees 2θ (±0.2 degrees 2θ).

In some embodiments, crystalline Form I of the maleate salt of Compound 1 is characterized by an XRPD pattern comprising seven peaks at 6.0, 13.3, 14.4, 17.2, 18.2, 19.2, and 26.5 degrees 2θ (±0.2 degrees 2θ).

In some embodiments, crystalline Form I of the maleate salt of Compound 1 is characterized by an XRPD pattern comprising eight peaks at 6.0, 13.3, 14.4, 17.2, 18.2, 19.2, 19.6, and 26.5 degrees 2θ (±0.2 degrees 2θ).

In some embodiments, crystalline Form I of the maleate salt of Compound 1 is characterized by an XRPD pattern comprising nine peaks at 6.0, 13.3, 14.4, 17.2, 18.2, 19.2, 19.6, 24.6, and 26.5 degrees 2θ (±0.2 degrees 2θ).

In some embodiments, crystalline Form I of the maleate salt of Compound 1 is characterized by a TGA thermogram substantially as shown in FIG. 26.

In some embodiments, crystalline Form I of the maleate salt of Compound 1 is characterized by weight loss of about 0.4% from 25° C. to 125° C. as determined by TGA.

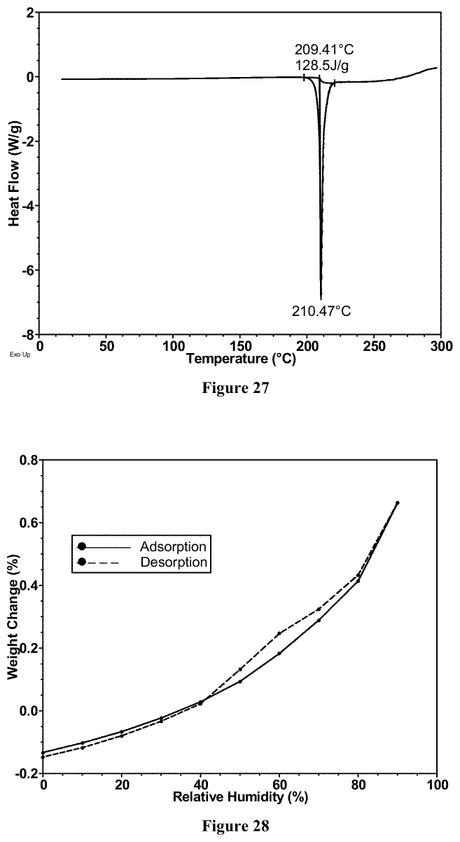

In some embodiments, crystalline Form I of the maleate salt of Compound 1 is characterized by a DSC thermogram substantially as shown in FIG. 27.

In some embodiments, crystalline Form I of the phosphate salt of Compound 1 is characterized by a DSC thermogram having a melting onset of about 209° C.

In some embodiments, crystalline Form I of the maleate salt of Compound 1 is characterized by a DVS sorption isotherm substantially as shown in FIG. 28.

In some embodiments, crystalline Form I of the maleate salt of Compound 1 is characterized by about 0.7% water uptake over a range of 0% to 90% RH at 25° C. as determined by DVS.

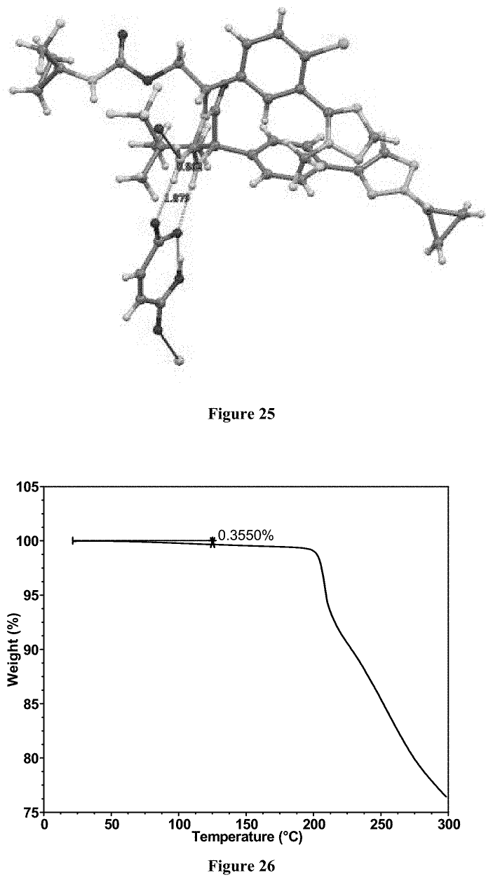

In some embodiments, crystalline Form I of the maleate salt of Compound 1 has an SCXRD molecular representation substantially as shown in FIG. 25.

In some embodiments, the crystalline Form I of the maleate salt of Compound 1 has an SCXRD molecular representation substantially as shown in Table 8.

In some embodiments, the crystalline Form I of the maleate salt of Compound 1 is characterized by a unit cell as determined by single crystal X-ray crystallography (SCXRD) of the following dimensions: a=15.57340(12) Å; b=10.23151(7) Å; c=27.7130(2) Å; α=90°; β=106.2880(8)°; and γ=90°.

### Compound 1 Succinate Salt

In some embodiments, Compound 1 can be isolated as a succinate salt which can be amorphous or crystalline.

In some embodiments, the crystalline succinate salt of Compound 1 is crystalline Form I.

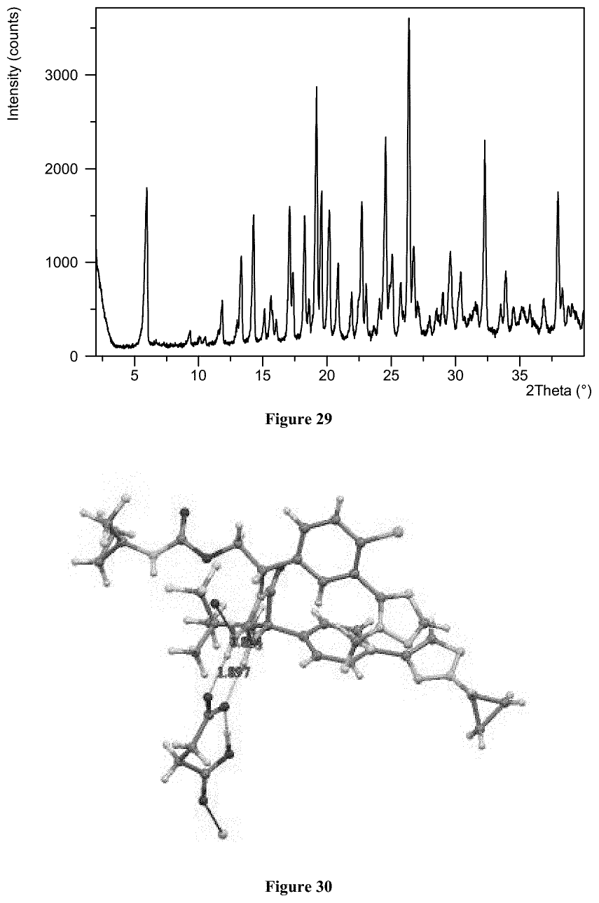

In some embodiments, crystalline Form I of the succinate salt of Compound 1 is characterized by an XRPD pattern substantially as shown in FIG. 29.

In some embodiments, crystalline Form I of the succinate salt of Compound 1 is characterized by an XRPD pattern comprising one peak, in terms of 2-theta±0.2°, selected from 6.0°, 13.3°, 14.3°, 17.1°, 18.3°, 19.2°, 19.6°, 24.6°, and 26.4°.

In some embodiments, crystalline Form I of the succinate salt of Compound 1 is characterized by an XRPD pattern comprising two peaks, in terms of 2-theta±0.2°, selected from 6.0°, 13.3°, 14.3°, 17.1°, 18.3°, 19.2°, 19.6°, 24.6°, and 26.4°.

In some embodiments, crystalline Form I of the succinate salt of Compound 1 is characterized by an XRPD pattern comprising three peaks, in terms of 2-theta±0.2°, selected from 6.0°, 13.3°, 14.3°, 17.1°, 18.3°, 19.2°, 19.6°, 24.6°, and 26.4°.

In some embodiments, crystalline Form I of the succinate salt of Compound 1 is characterized by an XRPD pattern comprising four peaks, in terms of 2-theta±0.2°, selected from 6.0°, 13.3°, 14.3°, 17.1°, 18.3°, 19.2°, 19.6°, 24.6°, and 26.4°.

In some embodiments, crystalline Form I of the succinate salt of Compound 1 is characterized by an XRPD pattern comprising five peaks, in terms of 2-theta±0.2°, selected from 6.0°, 13.3°, 14.3°, 17.1°, 18.3°, 19.2°, 19.6°, 24.6°, and 26.4°.

In some embodiments, crystalline Form I of the succinate salt of Compound 1 is characterized by an XRPD pattern comprising six peaks, in terms of 2-theta±0.2°, selected from 6.0°, 13.3°, 14.3°, 17.1°, 18.3°, 19.2°, 19.6°, 24.6°, and 26.4°.

In some embodiments, crystalline Form I of the succinate salt of Compound 1 is characterized by an XRPD pattern comprising seven peaks, in terms of 2-theta±0.2°, selected from 6.0°, 13.3°, 14.3°, 17.1°, 18.3°, 19.2°, 19.6°, 24.6°, and 26.4°.

In some embodiments, crystalline Form I of the succinate salt of Compound 1 is characterized by an XRPD pattern comprising eight peaks, in terms of 2-theta±0.2°, selected from 6.0°, 13.3°, 14.3°, 17.1°, 18.3°, 19.2°, 19.6°, 24.6°, and 26.4°.

In some embodiments, crystalline Form I of the succinate salt of Compound 1 is characterized by an XRPD pattern comprising three peaks at 6.0, 19.2, and 26.4 degrees 2θ (±0.2 degrees 2θ).

In some embodiments, crystalline Form I of the succinate salt of Compound 1 is characterized by an XRPD pattern comprising four peaks at 6.0, 13.3, 19.2, and 26.4 degrees 2θ (±0.2 degrees 2θ).

In some embodiments, crystalline Form I of the succinate salt of Compound 1 is characterized by an XRPD pattern comprising five peaks at 6.0, 13.3, 14.3, 19.2, and 26.4 degrees 2θ (±0.2 degrees 2θ).

In some embodiments, crystalline Form I of the succinate salt of Compound 1 is characterized by an XRPD pattern comprising six peaks at 6.0, 13.3, 14.3, 19.2, 24.6, and 26.4 degrees 2θ (±0.2 degrees 2θ).

In some embodiments, crystalline Form I of the succinate salt of Compound 1 is characterized by an XRPD pattern comprising seven peaks at 6.0, 13.3, 14.3, 17.1, 19.2, 24.6, and 26.4 degrees 2θ (±0.2 degrees 2θ).

In some embodiments, crystalline Form I of the succinate salt of Compound 1 is characterized by an XRPD pattern comprising eight peaks at 6.0, 13.3, 14.3, 17.1, 18.3, 19.2, 24.6, and 26.4 degrees 2θ (±0.2 degrees 2θ).

In some embodiments, crystalline Form I of the succinate salt of Compound 1 is characterized by an XRPD pattern comprising nine peaks at 6.0, 13.3, 14.3, 17.1, 18.3, 19.2, 19.6, 24.6, and 26.4 degrees 2θ (±0.2 degrees 2θ).

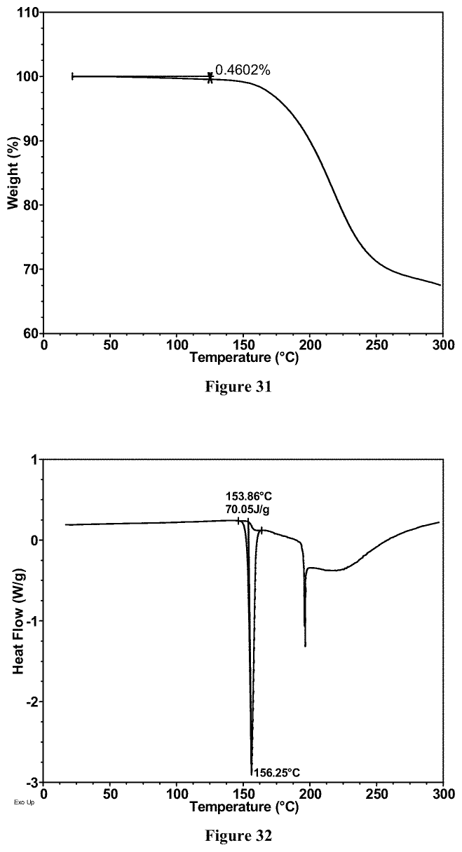

In some embodiments, crystalline Form I of the succinate salt of Compound 1 is characterized by a TGA thermogram substantially as shown in FIG. 31.

In some embodiments, crystalline Form I of the succinate salt of Compound 1 is characterized by weight loss of about 0.5% from 25° C. to 125° C. as determined by TGA.

In some embodiments, crystalline Form I of the succinate salt of Compound 1 is characterized by a DSC thermogram substantially as shown in FIG. 32.

In some embodiments, crystalline Form I of the succinate salt of Compound 1 is characterized by a DSC thermogram having a melting onset of about 154° C.

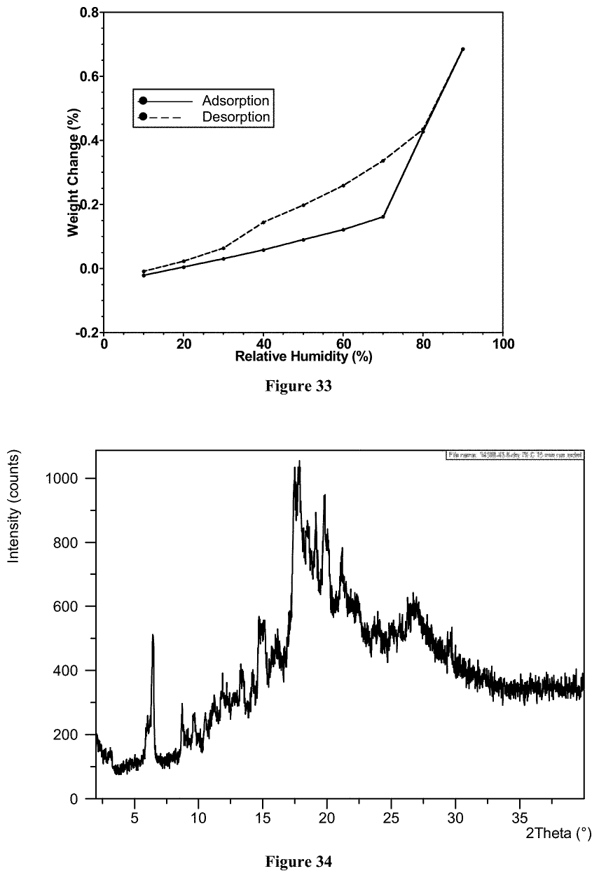

In some embodiments, crystalline Form I of the succinate salt of Compound 1 is characterized by a DVS sorption isotherm substantially as shown in FIG. 33.

In some embodiments, crystalline Form I of the succinate salt of Compound 1 is characterized by about 0.7% water uptake over a range of 10% to 90% RH at 25° C. as determined by DVS.

In some embodiments, crystalline Form I of the succinate salt of Compound 1 has an SCXRD molecular representation substantially as shown in FIG. 30.

In some embodiments, the crystalline Form I of the succinate salt of Compound 1 has an SCXRD molecular representation substantially as shown in Table 10.

In some embodiments, the crystalline Form I of the succinate salt of Compound 1 has) a unit cell as determined by single crystal X-ray crystallography of the following dimensions: a=27.6596(3) Å; b=10.22294(8) Å; c=15.57453(14) Å; α=90°; β=106.2803(10)°; and γ=90°.

### Compound 1 Hemisuccinate Salt

In some embodiments, Compound 1 can be isolated as a hemisuccinate salt which can be amorphous or crystalline.

In some embodiments, the crystalline hemisuccinate salt of Compound 1 is crystalline

Form I.

In some embodiments, crystalline Form I of the hemisuccinate salt of Compound 1 is characterized by an XRPD pattern substantially as shown in FIG. 34.

In some embodiments, crystalline Form I of the hemisuccinate salt of Compound 1 is characterized by an XRPD pattern comprising one peak, in terms of 2-theta±0.2°, selected from 6.5°, 8.7°, 15.1°, 17.4°, 17.9°, 18.5°, 19.1°, 19.8°, and 21.1°.

In some embodiments, crystalline Form I of the hemisuccinate salt of Compound 1 is characterized by an XRPD pattern comprising two peaks, in terms of 2-theta±0.2°, selected from 6.5°, 8.7°, 15.1°, 17.4°, 17.9°, 18.5°, 19.1°, 19.8°, and 21.1°.

In some embodiments, crystalline Form I of the hemisuccinate salt of Compound 1 is characterized by an XRPD pattern comprising three peaks, in terms of 2-theta±0.2°, selected from 6.5°, 8.7°, 15.1°, 17.4°, 17.9°, 18.5°, 19.1°, 19.8°, and 21.1°.

In some embodiments, crystalline Form I of the hemisuccinate salt of Compound 1 is characterized by an XRPD pattern comprising four peaks, in terms of 2-theta±0.2°, selected from 6.5°, 8.7°, 15.1°, 17.4°, 17.9°, 18.5°, 19.1°, 19.8°, and 21.1°.

In some embodiments, crystalline Form I of the hemisuccinate salt of Compound 1 is characterized by an XRPD pattern comprising five peaks, in terms of 2-theta±0.2°, selected from 6.5°, 8.7°, 15.1°, 17.4°, 17.9°, 18.5°, 19.1°, 19.8°, and 21.1°.

In some embodiments, crystalline Form I of the hemisuccinate salt of Compound 1 is characterized by an XRPD pattern comprising six peaks, in terms of 2-theta±0.2°, selected from 6.5°, 8.7°, 15.1°, 17.4°, 17.9°, 18.5°, 19.1°, 19.8°, and 21.1°.

In some embodiments, crystalline Form I of the hemisuccinate salt of Compound 1 is characterized by an XRPD pattern comprising seven peaks, in terms of 2-theta±0.2°, selected from 6.5°, 8.7°, 15.1°, 17.4°, 17.9°, 18.5°, 19.1°, 19.8°, and 21.1°.

In some embodiments, crystalline Form I of the hemisuccinate salt of Compound 1 is characterized by an XRPD pattern comprising eight peaks, in terms of 2-theta±0.2°, selected from 6.5°, 8.7°, 15.1°, 17.4°, 17.9°, 18.5°, 19.1°, 19.8°, and 21.1°.

In some embodiments, crystalline Form I of the hemisuccinate salt of Compound 1 is characterized by an XRPD pattern comprising peaks at 6.5, 17.9, and 19.8 degrees 2θ (±0.2 degrees 2θ).

In some embodiments, crystalline Form I of the hemisuccinate salt of Compound 1 is characterized by an XRPD pattern comprising peaks at 6.5, 8.7, 17.9, and 19.8 degrees 2θ (±0.2 degrees 2θ).

In some embodiments, crystalline Form I of the hemisuccinate salt of Compound 1 is characterized by an XRPD pattern comprising peaks at 6.5, 8.7, 17.4, 17.9, and 19.8 degrees 2θ (±0.2 degrees 2θ).

In some embodiments, crystalline Form I of the hemisuccinate salt of Compound 1 is characterized by an XRPD pattern comprising peaks at 6.5, 8.7, 17.4, 17.9, 19.8, and 21.1 degrees 2θ (±0.2 degrees 2θ).

In some embodiments, crystalline Form I of the hemisuccinate salt of Compound 1 is characterized by an XRPD pattern comprising peaks at 6.5, 8.7, 15.1, 17.4, 17.9, 19.8, and 21.1.

In some embodiments, crystalline Form I of the hemisuccinate salt of Compound 1 is characterized by an XRPD pattern comprising peaks at 6.5, 8.7, 15.1, 17.4, 17.9, 18.5, 19.8, and 21.1 degrees 2θ (±0.2 degrees 2θ).

In some embodiments, crystalline Form I of the hemisuccinate salt of Compound 1 is characterized by an XRPD pattern comprising peaks at 6.5, 8.7, 15.1, 17.4, 17.9, 18.5, 19.1, 19.8, and 21.1 degrees 2θ (±0.2 degrees 2θ).

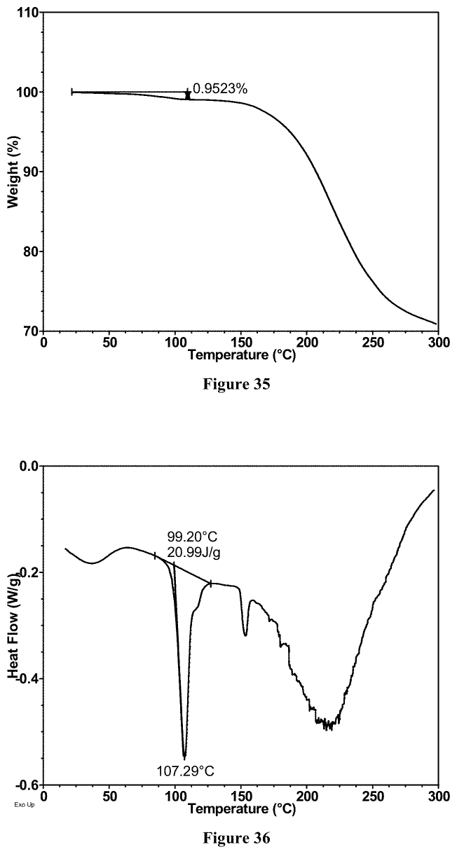

In some embodiments, crystalline Form I of the hemisuccinate salt of Compound 1 is characterized by a TGA thermogram substantially as shown in FIG. 35.

In some embodiments, crystalline Form I of the hemisuccinate salt of Compound 1 is characterized by weight loss of about 1.0% from 25° C. to 110° C. as determined by TGA.

In some embodiments, crystalline Form I of the hemisuccinate salt of Compound 1 is characterized by a DSC thermogram substantially as shown in FIG. 36.

In some embodiments, crystalline Form I of the hemisuccinate salt of Compound 1 is characterized by a DSC thermogram having a melting onset of about 99° C.

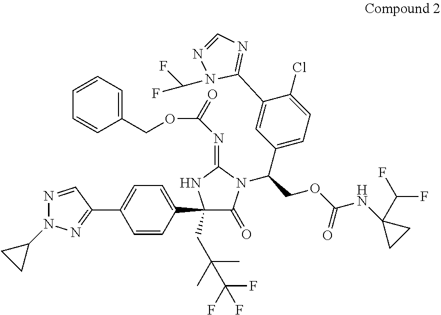 [SMILES: CC(C)(C[C@]1(c2ccc(-c3cnn(C4CC4)n3)cc2)N/C(=N\C(=O)OCc2ccccc2)N([C@H](COC(=O)NC2(C(F)F)CC2)c2ccc(Cl)c(-c3ncnn3C(F)F)c2)C1=O)C(F)(F)F]

Also provided herein are methyl isobutyl ketone (MIBK) and 2-butanone (MEK) solvate forms of Compound 2 (benzyl ((R,E)-1-((S)-1-(4-chloro-3-(1-(difluoromethyl)-1H-1,2,4-triazol-5-yl)phenyl)-2-(((1-(difluoromethyl)cyclopropyl)carbamoyl)oxy)ethyl)-4-(4-(2-cyclopropyl-2H-1,2,3-triazol-4-yl)phenyl)-5-oxo-4-(3,3,3-trifluoro-2,2-dimethylpropyl)imidazolidin-2-ylidene)carbamate).

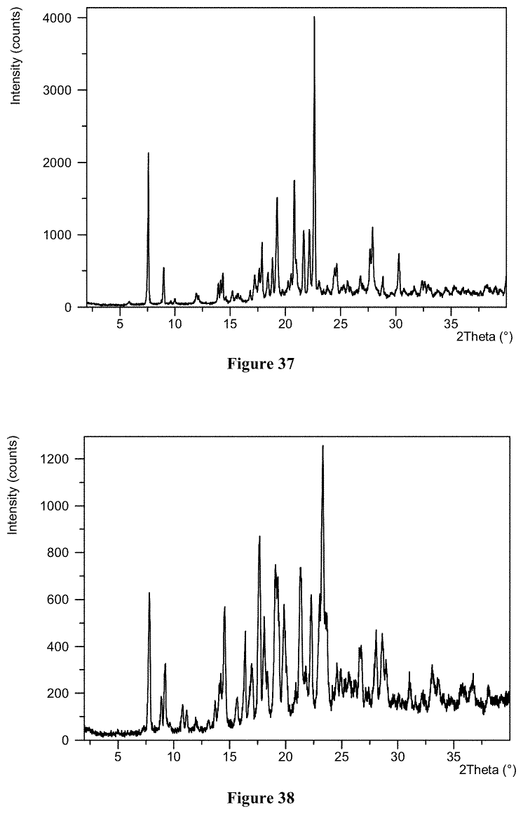

In some embodiments, the MIBK solvate form of Compound 2 is characterized by an XRPD pattern substantially as shown in FIG. 37.

In some embodiments, the MEK solvate form of Compound 2 is characterized by an XRPD pattern substantially as shown in FIG. 38.

Unless defined otherwise, all technical and scientific terms used herein have the same meaning as commonly understood by one of ordinary skill in the art.

When trade names are used herein, it is intended to independently include the tradename product and the active pharmaceutical ingredient(s) of the tradename product.

As used herein and in the appended claims, the singular forms “a” and “an”, and “the” include plural referents unless the context clearly dictates otherwise. Thus, e.g., reference to “the compound” includes a plurality of such compounds and reference to “the assay” includes reference to one or more assays, and so forth.

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” refers to a salt of a compound that is pharmaceutically acceptable and that possesses (or can be converted to a form that possesses) the desired pharmacological activity of the parent compound.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like.

As used herein, “treatment” or “treating” is an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and/or c) relieving the disease or condition, e.g., causing the regression of clinical symptoms, ameliorating the disease state, delaying the progression of the disease, increasing the quality of life, and/or prolonging survival.

As used herein, “delaying” development of a disease or condition means to defer, hinder, slow, retard, stabilize and/or postpone development of the disease or condition. This delay can be of varying lengths of time, depending on the history of the disease and/or subject being treated. As is evident to one skilled in the art, a sufficient or significant delay can, in effect, encompass prevention, in that the subject does not develop the disease or condition. For example, a method that “delays” development of AIDS is a method that reduces the probability of disease development in a given time frame and/or reduces extent of the disease in a given time frame, when compared to not using the method. Such comparisons may be based on clinical studies, using a statistically significant number of subjects. For example, the development of AIDS can be detected using known methods, such as confirming a subject's HIV⁺ status and assessing the subject's T-cell count or other indication of AIDS development, such as extreme fatigue, weight loss, persistent diarrhea, high fever, swollen lymph nodes in the neck, armpits or groin, or presence of an opportunistic condition that is known to be associated with AIDS (e.g., a condition that is generally not present in subjects with functioning immune systems but does occur in AIDS patients). Development may also refer to disease progression that may be initially undetectable and includes occurrence, recurrence and onset.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject (e.g., administration of a therapeutic substance to a subject in the absence of detectable infectious agent (e.g., virus) in the subject). The subject may be an individual at risk of developing the disease or disorder, such as an individual who has one or more risk factors known to be associated with development or onset of the disease or disorder. Thus, the term “preventing HIV infection” refers to administering to a subject who does not have a detectable HIV infection an anti-HIV therapeutic substance. It is understood that the subject for anti-HIV preventative therapy may be an individual at risk of contracting the HIV virus. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

As used herein, an “at risk” individual is an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

As used herein, the term “therapeutically effective amount” or “effective amount” refers to an amount that is effective to elicit the desired biological or medical response, including the amount of a compound that, when administered to a subject for treating a disease, is sufficient to effect such treatment for the disease or to an amount that is effective to protect against the contracting or onset of a disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment outcome. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

In addition, abbreviations as used herein have respective meanings as follows:

2-MeTHF 2-methyl tetrahydrofuran

BE Butyl ether

CPME Cyclopentyl methyl ether

DCM Dichloromethane

DSC Differential scanning calorimetry

DVS Dynamic vapor sorption

eq. Equivalents

EtOAc Ethyl acetate

EtOH Ethanol

g Gram

h/hr Hour

IPA Isopropanol

IPE Diisopropyl ether

IPAc/iPrOAc Isopropyl acetate

kV Kilovolts

MEK Methyl ethyl ketone

MeOH Methanol

MIBK Methyl iso-butyl ketone

mA Milliamps

mg Milligram

min Minute

mL/ml Milliliter

MTBE Methyl tert-butyl ether

n-BuOAc Butyl acetate

NMR Nuclear magnetic resonance

RH Relative humidity

RT Room temperature

s Second

SCXD Single crystal X-ray diffraction

TGA Thermogravimetric analysis

THF Tetrahydrofuran

u/v Volume to volume

wt Weight

wt/wt Weight to weight

XRPD X-ray powder diffraction

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

“Diastereoisomers” are stereoisomers that have at least two asymmetric atoms, but which are not mirror-images of each other.

The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds and salts described herein contain one or more asymmetric centers and/or hindered rotation about a bond axis and may thus give rise to enantiomers, diastereomers, and other stereoisomeric forms that may be defined, in terms of absolute stereochemistry, as (R)- or (S)-. The present disclosure is meant to include all such possible isomers, including racemic mixtures, scalemic mixtures, diastereomeric mixtures, optically pure forms and intermediate mixtures. Optically active (R)- and (S)-isomers may be prepared using chiral synthons or chiral reagents, or resolved using conventional techniques.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

It is understood by one skilled in the art that this disclosure also includes any salt disclosed herein may be enriched at any or all atoms above naturally occurring isotopic ratios with one or more isotopes such as, but not limited to, deuterium (²H or D).

Disclosed are also pharmaceutically acceptable salts and crystalline forms in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such salts and crystalline forms may increase resistance to metabolism, and thus may be useful for increasing the half-life of the compounds when administered to a mammal. See, e.g., Foster, “Deuterium Isotope Effects in Studies of Drug Metabolism”, Trends Pharmacol. Sci., 5(12):524-527 (1984). Such salts and crystalline forms are synthesized by means well known in the art, for example by employing starting materials in which one or more hydrogen atoms have been replaced by deuterium.

Examples of isotopes that can be incorporated into the disclosed salts and crystalline forms also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁵Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled salts can generally be prepared by conventional techniques known to those skilled in the art or by processes analogous to those described in the Examples as set out below using an appropriate isotopically-labeled reagent in place of the non-labeled reagent previously employed.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s).

In a preferred embodiment, the current disclosure relates to the use of the pharmaceutically acceptable salts and crystalline forms of the invention in treating a Retroviridae viral infection including an infection caused by the HIV virus comprising administering a therapeutically effective amount of the pharmaceutically acceptable salt or crystalline form to a subject in need thereof.

In a preferred embodiment, the current disclosure relates to the use of the pharmaceutically acceptable salts and crystalline forms of the invention in treating a Retroviridae viral infection including an infection caused by the HIV virus comprising administering a therapeutically effective amount of the pharmaceutically acceptable salt or crystalline form to a subject in need thereof.

It is a desirable goal to discover a compound, a pharmaceutically acceptable salt or crystalline form thereof that has good physical and/or chemical stability. An increase in overall stability of a compound, salt or crystalline form can provide an increase in circulation time in the body. With less degradation, a stable compound, salt or crystalline form can be administered in lower doses and still maintain efficacy. Also, with less degradation, there is less concern about by-products from degradation of a compound, salt or crystalline form.

It is a desirable goal to discover a compound, a pharmaceutically acceptable salt or crystalline form thereof that has improved pharmacokinetic and/or pharmacodynamic profiles and long half-life. It is advantageous for a drug to have a moderate or low clearance and a long half-life, as this can lead to a good bioavailability and high exposure in systemic exposure. Reducing the clearance and increasing half-life time of a compound, salt or crystalline form could reduce the daily dose required for efficacy and therefore give a better efficacy and safety profile. Thus, improved pharmacokinetic and/or pharmacodynamic profiles and long half-life can provide for better patient compliance.

### Methods of Use

In some embodiments, the pharmaceutically acceptable salts or crystalline forms disclosed herein are used for preventing an HIV infection in a subject. In some embodiments, the pharmaceutically acceptable salts or crystalline forms disclosed herein are used for preventing an HIV infection in a subject at risk for infection. In some embodiments, the pharmaceutically acceptable salts or crystalline forms disclosed herein are used for pre-exposure prophylaxis (PrEP) to reduce the risk of sexually acquired HIV-1. It is believed that the pharmaceutically acceptable salts or crystalline forms disclosed herein are active against major HIV-1 mutants selected by clinical Protease Inhibitors (PIs), nucleoside reverse transcriptase inhibitors (NRTIs), Non-Nucleoside Reverse Transcriptase Inhibitors (NNRTIs), and Integrase inhibitors (INSTIs).

In certain embodiments, a method for treating or preventing an HIV infection in a subject (e.g., a human), comprising administering a pharmaceutically acceptable salt of Compound 1, or a crystalline form thereof, to the subject is disclosed.

In some embodiments, a method for treating or preventing an HIV infection in a subject (e.g., a human), comprising administering a pharmaceutically acceptable salt of Compound 1, or a crystalline form thereof, to the subject is disclosed.

In certain embodiments, a method for inhibiting the replication of the HIV virus, treating AIDS or delaying the onset of AIDS in a subject (e.g., a human), comprising administering a pharmaceutically acceptable salt of Compound 1, or a crystalline form thereof, to the subject is disclosed.

In some embodiments, a method for inhibiting the replication of the HIV virus, treating AIDS or delaying the onset of AIDS in a subject (e.g., a human), comprising administering a pharmaceutically acceptable salt of Compound 1, or a crystalline form thereof, to the subject is disclosed.

In certain embodiments, a method for preventing an HIV infection in a subject (e.g., a human), comprising administering a pharmaceutically acceptable salt of Compound 1, or a crystalline form thereof, to the subject is disclosed. In certain embodiments, the subject is at risk of contracting the HIV virus, such as a subject who has one or more risk factors known to be associated with contracting the HIV virus.

In some embodiments, a method for preventing an HIV infection in a subject (e.g., a human), comprising administering a therapeutically effective amount of a pharmaceutically acceptable salt of Compound 1, or a crystalline form thereof, to the subject is disclosed. In certain embodiments, the subject is at risk of contracting the HIV virus, such as a subject who has one or more risk factors known to be associated with contracting the HIV virus.

In certain embodiments, a method for treating an HIV infection in a subject (e.g., a human), comprising administering a pharmaceutically acceptable salt of Compound 1, or a crystalline form thereof, to the subject is disclosed.

In some embodiments, a method for treating an HIV infection in a subject (e.g., a human), comprising administering a pharmaceutically acceptable salt of Compound 1, or a crystalline form thereof, to the subject is disclosed.

In certain embodiments, a method for treating an HIV infection in a subject (e.g., a human), comprising administering to the subject in need thereof a therapeutically effective amount of a pharmaceutically acceptable salt of Compound 1, or a crystalline form thereof, in combination with a therapeutically effective amount of one or more (e.g., one, two, three, or four; or one or two; or one to three; or one to four) additional therapeutic agents selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof is disclosed. In certain embodiments, a method for treating an HIV infection in a subject (e.g., a human), comprising administering to the subject in need thereof a therapeutically effective amount of a pharmaceutically acceptable salt of Compound 1, or a crystalline form thereof, in combination with a therapeutically effective amount of one or more (e.g., one, two, three, or four; or one or two; or one to three; or one to four) additional therapeutic agents selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof is disclosed. In some embodiments, a method for treating an HIV infection in a subject (e.g., a human), comprising administering to the subject in need thereof a therapeutically effective amount of a pharmaceutically acceptable salt of Compound 1, or a crystalline form thereof, in combination with a therapeutically effective amount of one or more (e.g., one, two, three, or four; or one or two; or one to three; or one to four) additional therapeutic agents selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof is disclosed. In certain embodiments, a method for treating an HIV infection in a subject (e.g., a human), comprising administering to the subject in need thereof a therapeutically effective amount of a pharmaceutically acceptable salt of Compound 1, or a crystalline form thereof, in combination with a therapeutically effective amount of one or more (e.g., one, two, three, or four; or one or two; or one to three; or one to four) additional therapeutic agents selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof is disclosed.

In certain embodiments, a method for treating an HIV infection in a subject (e.g., a human), comprising administering to the subject in need thereof a therapeutically effective amount of a pharmaceutically acceptable salt of Compound 1, or a crystalline form thereof, in combination with a therapeutically effective amount of one or more (e.g., one, two, three, or four; or one or two; or one to three; or one to four) additional therapeutic agents selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, and HIV nucleoside reverse transcriptase translocation inhibitors.

In certain embodiments, a pharmaceutically acceptable salt of Compound 1, or a crystalline form thereof, for use in medical therapy of an HIV infection (e.g. HIV-1 or the replication of the HIV virus (e.g. HIV-1) or AIDS or delaying the onset of AIDS in a subject (e.g., a human)) is disclosed.

In some embodiments, a pharmaceutically acceptable salt of Compound 1, or a crystalline form thereof, for use in medical therapy of an HIV infection (e.g. HIV-1 or the replication of the HIV virus (e.g. HIV-1) or AIDS or delaying the onset of AIDS in a subject (e.g., a human)) is disclosed.

In certain embodiments, a pharmaceutically acceptable salt of Compound 1, or a crystalline form thereof, for use in the manufacture of a medicament for treating an HIV infection or the replication of the HIV virus or AIDS or delaying the onset of AIDS in a subject (e.g., a human) is disclosed. One embodiment relates to a pharmaceutically acceptable salt of Compound 1, or a crystalline form thereof, for use in the prophylactic or therapeutic treatment of an HIV infection or AIDS or for use in the therapeutic treatment or delaying the onset of AIDS.

In some embodiments, a pharmaceutically acceptable salt of Compound 1, or a crystalline form thereof for use in the manufacture of a medicament for treating an HIV infection or the replication of the HIV virus or AIDS or delaying the onset of AIDS in a subject (e.g., a human) is disclosed. One embodiment relates to a pharmaceutically acceptable salt of Compound 1, or a crystalline form thereof, for use in the prophylactic or therapeutic treatment of an HIV infection or AIDS or for use in the therapeutic treatment or delaying the onset of AIDS.

In certain embodiments, the use of a pharmaceutically acceptable salt of Compound 1, or a crystalline form thereof, for the manufacture of a medicament for an HIV infection in a subject (e.g., a human) is disclosed. In certain embodiments, a pharmaceutically acceptable salt of Compound 1, or a crystalline form thereof, for use in the prophylactic or therapeutic treatment of an HIV infection is disclosed.

In some embodiments, the use of a pharmaceutically acceptable salt of Compound 1, or a crystalline form thereof, for the manufacture of a medicament for an HIV infection in a subject (e.g., a human) is disclosed. In certain embodiments, a pharmaceutically acceptable salt of Compound 1, or a crystalline form thereof, for use in the prophylactic or therapeutic treatment of an HIV infection is disclosed.

In certain embodiments, in the methods of use, the administration is to a subject (e.g., a human) in need of the treatment. In certain embodiments, in the methods of use, the administration is to a subject (e.g., a human) who is at risk of developing AIDS.

Disclosed herein is a pharmaceutically acceptable salt of Compound 1, or a crystalline form thereof, for use in therapy. In one embodiment, the pharmaceutically acceptable salt of Compound 1, or a crystalline form thereof, is for use in a method of treating an HIV infection or the replication of the HIV virus or AIDS or delaying the onset of AIDS in a subject (e.g., a human).

In some embodiments, disclosed herein is a pharmaceutically acceptable salt of Compound 1, or a crystalline form thereof, for use in therapy. In some embodiments, the pharmaceutically acceptable salt of Compound 1, or a crystalline form thereof, is for use in a method of treating an HIV infection or the replication of the HIV virus or AIDS or delaying the onset of AIDS in a subject (e.g., a human).

Also disclosed herein is a pharmaceutically acceptable salt of Compound 1, or a crystalline form thereof, for use in a method of treating or preventing HIV infection in a subject in need thereof. In certain embodiments, a pharmaceutically acceptable salt of Compound 1, or a crystalline form thereof, for use in a method of treating HIV infection in a subject in need thereof is provided. In certain embodiments, the subject in need thereof is a human who has been infected with HIV. In certain embodiments, the subject in need thereof is a human who has been infected with HIV but who has not developed AIDS. In certain embodiments, the subject in need thereof is a subject at risk for developing AIDS. In certain embodiments, the subject in need thereof is a human who has been infected with HIV and who has developed AIDS.

In some embodiments, disclosed herein is a pharmaceutically acceptable salt of Compound 1, or a crystalline form thereof, for use in a method of treating or preventing HIV infection in a subject in need thereof. In certain embodiments, a pharmaceutically acceptable salt of Compound 1, or a crystalline form thereof, for use in a method of treating HIV infection in a subject in need thereof is provided. In certain embodiments, the subject in need thereof is a human who has been infected with HIV. In certain embodiments, the subject in need thereof is a human who has been infected with HIV but who has not developed AIDS. In certain embodiments, the subject in need thereof is a subject at risk for developing AIDS. In certain embodiments, the subject in need thereof is a human who has been infected with HIV and who has developed AIDS.

In one embodiment, a pharmaceutically acceptable salt of Compound 1, or a crystalline form thereof, in combination with one or more (e.g. one, two, three, or four; or one or two; or one to three; or one to four) additional therapeutic agents as described herein for use in a method of treating or preventing HIV infection in a subject in need thereof is provided. In one embodiment, said additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof. In one embodiment, said additional therapeutic agents are selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof.

In some embodiments, a pharmaceutically acceptable salt of Compound 1, or a crystalline form thereof, in combination with one or more (e.g. one, two, three, or four; or one or two; or one to three; or one to four) additional therapeutic agents as described herein for use in a method of treating or preventing HIV infection in a subject in need thereof is provided. In one embodiment, said additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof. In one embodiment, said additional therapeutic agents are selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof.

In one embodiment, a pharmaceutically acceptable salt of Compound 1, or a cocrystal or crystalline form thereof, in combination with a first additional therapeutic agent selected from the group consisting of tenofovir alafenamide fumarate, tenofovir alafenamide, and tenofovir alafenamide hemifumarate, and a second additional therapeutic agent, wherein the second additional therapeutic agent is emtricitabine, is provided for use in a method of treating or preventing HIV infection in a subject in need thereof. In a particular embodiment, a pharmaceutically acceptable salt of Compound 1, or a crystalline form thereof, in combination with a first additional therapeutic agent selected from the group consisting of tenofovir disoproxil fumarate, tenofovir disoproxil, and tenofovir disoproxil hemifumarate, and a second additional therapeutic agent, wherein the second additional therapeutic agent is emtricitabine, is provided for use in a method of treating or preventing HIV infection in a subject in need thereof.

In some embodiments, a pharmaceutically acceptable salt of Compound 1, or a crystalline form thereof, in combination with a first additional therapeutic agent selected from the group consisting of tenofovir alafenamide fumarate, tenofovir alafenamide, and tenofovir alafenamide hemifumarate, and a second additional therapeutic agent, wherein the second additional therapeutic agent is emtricitabine, is provided for use in a method of treating or preventing HIV infection in a subject in need thereof. In a particular embodiment, a pharmaceutically acceptable salt of Compound 1, or a crystalline form thereof, in combination with a first additional therapeutic agent selected from the group consisting of tenofovir disoproxil fumarate, tenofovir disoproxil, and tenofovir disoproxil hemifumarate, and a second additional therapeutic agent, wherein the second additional therapeutic agent is emtricitabine, is provided for use in a method of treating or preventing HIV infection in a subject in need thereof.

In some embodiments, a pharmaceutically acceptable salt of Compound 1, or a crystalline form thereof, is provided in combination with at least one additional therapeutic agent selected from the group consisting of:
(1) nucleoside reverse transcriptase translocation inhibitors (“NRTTIs”), such as 4′-Ethynyl-2-fluoro-2′-deoxyadenosine triphosphate (also known as MK-8591 and EFdA);
(2) nucleoside or nucleotide reverse transcriptase inhibitors (“NRTIs”), such as tenofovir alafenamide fumarate, tenofovir alafenamide, tenofovir alafenamide hemifumarate, GS-9131, and GS-9148;
(3) non-nucleoside or non-nucleotide reverse transcriptase inhibitors (“NNRTIs”), such as efavirenz, etravirine, rilpivirine, nevirapine, and delavirdine;
(4) protease Inhibitors (“PIs”), such as amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, and TMC-31091; and
(5) integrase strand transfer inhibitors (“INSTIs”), such as Bictegravir, cabotegravir, raltegravir, and dolutegravir.

In a particular embodiment, a pharmaceutically acceptable salt of Compound 1, or a crystalline form thereof, is provided for use to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP). Accordingly, in certain embodiments, methods for reducing the risk of acquiring HIV (e.g., HIV-1 and/or HIV-2) are provided. For example, methods for reducing the risk of acquiring HIV (e.g., HIV-1 and/or HIV-2) comprise administration of a pharmaceutically acceptable salt of Compound 1, or a crystalline form thereof. In certain embodiments, methods for reducing the risk of acquiring HIV (e.g., HIV-1 and/or HIV-2) comprise administration of a pharmaceutically acceptable salt of Compound 1, or a crystalline form thereof, in combination with one or more additional therapeutic agents. In certain embodiments, methods for reducing the risk of acquiring HIV (e.g., HIV-1 and/or HIV-2) comprise administration of a pharmaceutical composition comprising a therapeutically effective amount of a pharmaceutically acceptable salt of Compound 1, or a crystalline form thereof, and a pharmaceutically acceptable excipient.

In some embodiments, a pharmaceutically acceptable salt of Compound 1, or a crystalline form thereof, is provided for use to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP). Accordingly, in certain embodiments, methods for reducing the risk of acquiring HIV (e.g., HIV-1 and/or HIV-2) are provided. For example, methods for reducing the risk of acquiring HIV (e.g., HIV-1 and/or HIV-2) comprise administration of a pharmaceutically acceptable salt of Compound 1, or crystalline form thereof. In certain embodiments, methods for reducing the risk of acquiring HIV (e.g., HIV-1 and/or HIV-2) comprise administration of a pharmaceutically acceptable salt of Compound 1, or a crystalline form thereof, in combination with one or more additional therapeutic agents. In certain embodiments, methods for reducing the risk of acquiring HIV (e.g., HIV-1 and/or HIV-2) comprise administration of a pharmaceutical composition comprising a therapeutically effective amount of a pharmaceutically acceptable salt of Compound 1, or a crystalline form thereof, and a pharmaceutically acceptable excipient.

In certain embodiments, methods for reducing the risk of acquiring HIV (e.g., HIV-1 and/or HIV-2) comprise administration of a pharmaceutically acceptable salt of Compound 1, or a crystalline form thereof, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In some embodiments, methods for reducing the risk of acquiring HIV (e.g., HIV-1 and/or HIV-2) comprise administration of a pharmaceutically acceptable salt of Compound 1, or a crystalline form thereof, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In certain embodiments, the reduction in risk of acquiring HIV is at least about 40%, 50%, 60%, 70%, 80%, 90%, or 95%. In certain embodiments, the reduction in risk of acquiring HIV is at least about 75%. In certain embodiments, the reduction in risk of acquiring HIV is about 80%, 85%, or 90%.

In another embodiment, the use of a pharmaceutically acceptable salt of Compound 1, or a crystalline form thereof, for the manufacture of a medicament for the treatment of an HIV infection in a human being having or at risk of having the infection is disclosed.

In some embodiments, the use of a pharmaceutically acceptable salt of Compound 1, or a crystalline form thereof, for the manufacture of a medicament for the treatment of an HIV infection in a human being having or at risk of having the infection is disclosed.

Also disclosed herein is a pharmaceutically acceptable salt of Compound 1, or a crystalline form thereof, for use in the therapeutic treatment or delaying the onset of AIDS.

In some embodiments, disclosed herein is a pharmaceutically acceptable salt of Compound 1, or a crystalline form thereof, for use in the therapeutic treatment or delaying the onset of AIDS.

Also disclosed herein is a pharmaceutically acceptable salt of Compound 1, or a crystalline form thereof, for use in the prophylactic or therapeutic treatment of an HIV infection.

In some embodiments, disclosed herein is a pharmaceutically acceptable salt of Compound 1, or a crystalline form thereof, for use in the prophylactic or therapeutic treatment of an HIV infection.

In certain embodiments, a pharmaceutically acceptable salt of Compound 1, or a crystalline form thereof, can be used as a research tool.

### Routes of Administration

The pharmaceutically acceptable salt of Compound 1, or a crystalline form thereof, (also referred to herein as the active ingredient) can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with, for example, the condition of the recipient. In certain embodiments, the compounds, salts or crystalline forms disclosed can be dosed parenterally. In certain embodiments, the compounds, salts or crystalline forms disclosed can be dosed intravenous, subcutaneous, or intramuscular. In certain embodiments, the compounds, salts and crystalline forms disclosed are orally bioavailable and can be dosed orally.

In some embodiments, the pharmaceutically acceptable salt of Compound 1, or a crystalline form thereof, may be administered with a syringe suitable for administration of the compound. In some embodiments, the syringe is disposable. In some embodiments, the syringe is reusable. In some embodiments, the syringe is pre-filled with the pharmaceutically acceptable salt of Compound 1, or a crystalline form thereof.

In some embodiments, the pharmaceutically acceptable salt of Compound 1, or a crystalline form thereof, may be administered via injection, using an injection device. In some embodiments, the injection device is or includes a syringe, which can be employed manually, or as part of a syringe-containing injection device, such as, but not limited to, one with a needle safety shield. A wide variety of injection devices can be used, such as, for example and not limited to, a handheld or wearable autoinjector, a handheld or wearable manual injector, an on-body injector, a syrette, a jet injector, or a pen injector, each of which can be reusable or disposable.

In some embodiments, the pharmaceutically acceptable salt of Compound 1, or a crystalline form thereof, may be administered with an auto-injector comprising a syringe. In some embodiments, the syringe is disposable. In some embodiments, the syringe is reusable. In some embodiments, the syringe is pre-filled with the pharmaceutically acceptable salt of Compound 1, or a crystalline form thereof.

### Dosing Regimen

The pharmaceutically acceptable salt of Compound 1, or a crystalline form thereof, may be administered to a subject in accordance with an effective dosing regimen for a desired period of time or duration, such as at least about one day, at least about one week, at least about one month, at least about 2 months, at least about 3 months, at least about 4 months, at least about 6 months, or at least about 12 months or longer. In one variation, the salt or crystalline form is administered on a daily or intermittent schedule. In one variation, the salt or crystalline form is administered on a monthly schedule. In one variation, the salt or crystalline form is administered every two months. In one variation, the salt or crystalline form is administered every three months. In one variation, the salt or crystalline form is administered every four months. In one variation, the salt or crystalline form is administered every five months. In one variation, the salt or crystalline form is administered every 6 months.

In some embodiments, the pharmaceutically acceptable salt of Compound 1, or a crystalline form thereof, may be administered to a subject at least about one month, at least about 4 months, or at least about 6 months. In some embodiments, the pharmaceutically acceptable salt of Compound 1, or a crystalline form thereof, may be subcutaneously administered to a subject at least about one month. In some embodiments, the pharmaceutically acceptable salt of Compound 1, or a crystalline form thereof, may be subcutaneously or intramuscularly administered to a subject at least about 4 months, or at least about 6 months.

The dosage or dosing frequency of the pharmaceutically acceptable salt of Compound 1, or a crystalline form thereof, may be adjusted over the course of the treatment, based on the judgment of the administering physician.

In some embodiments, the dosage or dosing frequency of the pharmaceutically acceptable salt of Compound 1, or a crystalline form thereof, may be adjusted over the course of the treatment, based on the judgment of the administering physician.

The pharmaceutically acceptable salt of Compound 1, or a crystalline form thereof may be administered to a subject (e.g., a human) in an effective amount. In certain embodiments, the pharmaceutically acceptable salt of Compound 1, or a crystalline form thereof is administered once daily.

In some embodiments, the pharmaceutically acceptable salt of Compound 1, or a crystalline form thereof, may be administered to a subject (e.g., a human) in a therapeutically effective amount. In some embodiments, the pharmaceutically acceptable salt of Compound 1, or a crystalline form thereof, is administered once daily. In some embodiments, the pharmaceutically acceptable salt of Compound 1, or a crystalline form thereof, is administered monthly. In some embodiments, the pharmaceutically acceptable salt of Compound 1, or a crystalline form thereof, is administered every three months. In some embodiments, the pharmaceutically acceptable salt of Compound 1, or a crystalline form thereof, is administered every four months. In some embodiments, the pharmaceutically acceptable salt of Compound 1, or a crystalline form thereof, is administered every six months.

A pharmaceutically acceptable salt of Compound 1, or a crystalline form thereof, disclosed herein may be administered in a dosage amount that is effective. For example, the dosage amount can be from 1 mg to 1000 mg of compound. In certain embodiments, the dosage amount is about 1, 10, 20, 30, 40, 50, 60, 70, 80, 90, 95, 100, 105, 110, 120, 130, 140, or 150 mg of compound. In certain embodiments the dosage amount is about 100, 150, 200, 250, 300, 350, 400, 450, 500, 550, 600, 650, 700, 750, 800, 850, 900, 950, or 1000 mg.

In some embodiments, the pharmaceutically acceptable salt of Compound 1, or a crystalline form thereof, is administered in a once daily dose. In some embodiments, the pharmaceutically acceptable salt of Compound 1, or a crystalline form thereof, is administered in a once daily dose of about 1 mg.

In some embodiments, the pharmaceutically acceptable salt of Compound 1, or a crystalline form thereof, is administered monthly. In some embodiments, the pharmaceutically acceptable salt of Compound 1, or a crystalline form thereof, is administered monthly at a dose of about 100 mg.

In some embodiments, the pharmaceutically acceptable salt of Compound 1, or a crystalline form thereof, is administered every 6 months. In some embodiments, the pharmaceutically acceptable salt of Compound 1, or a crystalline form thereof, is administered every 6 months at a dose of about 600 mg.

### Combination Therapies

In certain embodiments, a method for treating or preventing an HIV infection in a human having or at risk of having the infection is provided, comprising administering to the human a therapeutically effective amount of a pharmaceutically acceptable salt of Compound 1, or a crystalline form thereof, disclosed herein in combination with a therapeutically effective amount of one or more (e.g., one, two, three, or four; or one or two; or one to three; or one to four) additional therapeutic agents. In one embodiment, a method for treating an HIV infection in a human having or at risk of having the infection is provided, comprising administering to the human a therapeutically effective amount of a pharmaceutically acceptable salt of Compound 1, or a crystalline form thereof, disclosed herein in combination with a therapeutically effective amount of one or more (e.g., one, two, three, or four; or one or two; or one to three; or one to four) additional therapeutic agents.

In some embodiments, a method for treating or preventing an HIV infection in a human having or at risk of having the infection is provided, comprising administering to the human a therapeutically effective amount of a pharmaceutically acceptable salt of Compound 1, or a crystalline form thereof, in combination with a therapeutically effective amount of one or more (e.g., one, two, three, or four; or one or two; or one to three; or one to four) additional therapeutic agents. In one embodiment, a method for treating an HIV infection in a human having or at risk of having the infection is provided, comprising administering to the human a therapeutically effective amount of a pharmaceutically acceptable salt of Compound 1, or a crystalline form thereof, in combination with a therapeutically effective amount of one or more (e.g., one, two, three, or four; or one or two; or one to three; or one to four) additional therapeutic agents.

In one embodiment, pharmaceutical compositions comprising a pharmaceutically acceptable salt of Compound 1, or a crystalline form thereof, in combination with one or more (e.g., one, two, three, or four; or one or two; or one to three; or one to four) additional therapeutic agents, and a pharmaceutically acceptable excipient are provided.

In some embodiments, pharmaceutical compositions comprising a pharmaceutically acceptable salt of Compound 1, or a crystalline form thereof, in combination with one or more (e.g., one, two, three, or four; or one or two; or one to three; or one to four) additional therapeutic agents, and a pharmaceutically acceptable excipient are provided.

In certain embodiments, the present disclosure provides a method for treating an HIV infection, comprising administering to a subject in need thereof a therapeutically effective amount of a pharmaceutically acceptable salt of Compound 1, or a crystalline form thereof, in combination with a therapeutically effective amount of one or more additional therapeutic agents which are suitable for treating an HIV infection.

In certain embodiments, the present disclosure provides a method for treating an HIV infection, comprising administering to a subject in need thereof a therapeutically effective amount of a pharmaceutically acceptable salt of Compound 1, or a crystalline form thereof, in combination with a therapeutically effective amount of one or more additional therapeutic agents which are suitable for treating an HIV infection.

In certain embodiments, a pharmaceutically acceptable salt of Compound 1, or a crystalline form thereof, disclosed herein is combined with one, two, three, four, or more additional therapeutic agents. In certain embodiments, a pharmaceutically acceptable salt of Compound 1, or a crystalline form thereof, disclosed herein is combined with one additional therapeutic agent. In certain embodiments, a pharmaceutically acceptable salt of Compound 1, or a crystalline form thereof, disclosed herein is combined with two additional therapeutic agents. In other embodiments, a pharmaceutically acceptable salt of Compound 1, or a crystalline form thereof, disclosed herein is combined with three additional therapeutic agents. In further embodiments, a pharmaceutically acceptable salt of Compound 1, or a crystalline form thereof, disclosed herein is combined with four additional therapeutic agents. The one, two, three, four, or more additional therapeutic agents can be different therapeutic agents selected from the same class of therapeutic agents, and/or they can be selected from different classes of therapeutic agents.

In some embodiments, a pharmaceutically acceptable salt of Compound 1, or a crystalline form thereof, disclosed herein is combined with one, two, three, four, or more additional therapeutic agents. In certain embodiments, a pharmaceutically acceptable salt of Compound 1, or a crystalline form thereof, disclosed herein is combined with one additional therapeutic agent. In certain embodiments, a pharmaceutically acceptable salt of Compound 1, or a crystalline form thereof, disclosed herein is combined with two additional therapeutic agents. In other embodiments, a pharmaceutically acceptable salt of Compound 1, or a crystalline form thereof, disclosed herein is combined with three additional therapeutic agents. In further embodiments, a pharmaceutically acceptable salt of Compound 1, or a crystalline form thereof, disclosed herein is combined with four additional therapeutic agents. The one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a pharmaceutically acceptable salt of Compound 1, or a crystalline form thereof, disclosed herein is administered with one or more additional therapeutic agents. Co-administration of a pharmaceutically acceptable salt of Compound 1, or a crystalline form thereof, disclosed herein with one or more additional therapeutic agents generally refers to simultaneous or sequential administration of a pharmaceutically acceptable salt of Compound 1, or a crystalline form thereof, and one or more additional therapeutic agents, such that therapeutically effective amounts of the pharmaceutically acceptable salt of Compound 1, or a crystalline form thereof, and the one or more additional therapeutic agents are both present in the body of the subject. When administered sequentially, the combination may be administered in two or more administrations.

In some embodiments, a pharmaceutically acceptable salt of Compound 1, or a crystalline form thereof, disclosed herein is administered with one or more additional therapeutic agents. Co-administration of a pharmaceutically acceptable salt of Compound 1, or a crystalline form thereof, disclosed herein with one or more additional therapeutic agents generally refers to simultaneous or sequential administration of the pharmaceutically acceptable salt of Compound 1, or a crystalline form thereof, and one or more additional therapeutic agents, such that therapeutically effective amounts of the a pharmaceutically acceptable salt of Compound 1, or a crystalline form thereof, and the one or more additional therapeutic agents are both present in the body of the subject. When administered sequentially, the combination may be administered in two or more administrations.

Co-administration includes administration of unit dosages of the pharmaceutically acceptable salt of Compound 1, or a crystalline form thereof, before or after administration of unit dosages of one or more additional therapeutic agents. For example, the pharmaceutically acceptable salt of Compound 1, or a crystalline form thereof, may be administered within seconds, minutes, or hours of the administration of the one or more additional therapeutic agents. In some embodiments, a unit dose of a pharmaceutically acceptable salt of Compound 1, or a crystalline form thereof, is administered first, followed within seconds or minutes by administration of a unit dose of one or more additional therapeutic agents. Alternatively, a unit dose of one or more additional therapeutic agents is administered first, followed by administration of a unit dose of a pharmaceutically acceptable salt of Compound 1, or a crystalline form thereof, within seconds or minutes. In other embodiments, a unit dose of a pharmaceutically acceptable salt of Compound 1, or a crystalline form thereof, is administered first, followed, after a period of hours (e.g., 1-12 hours), by administration of a unit dose of one or more additional therapeutic agents. In yet other embodiments, a unit dose of one or more additional therapeutic agents is administered first, followed, after a period of hours (e.g., 1-12 hours), by administration of a unit dose of a pharmaceutically acceptable salt of Compound 1, or a crystalline form thereof.

In certain embodiments, a pharmaceutically acceptable salt of Compound 1, or a crystalline form thereof, disclosed herein is combined with one or more additional therapeutic agents in a unitary dosage form for simultaneous administration to a subject. In certain embodiments, such a unitary dosage form can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. In certain embodiments, the compounds disclosed can be dosed parenterally. In certain embodiments, the unitary dosage form can be dosed intravenous, subcutaneous, or intramuscular. In certain embodiments, the unitary dosage form is orally bioavailable and can be dosed orally. In certain embodiments, the unitary dosage form can be a solid dosage form for oral administration.

The pharmaceutically acceptable salt of Compound 1, or a crystalline form thereof, disclosed herein in combination with one or more additional therapeutic agents can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. In certain embodiments, the pharmaceutically acceptable salt of Compound 1, or a crystalline form thereof, disclosed herein can be dosed parenterally. In certain embodiments, the pharmaceutically acceptable salt of Compound 1, or a crystalline form thereof, disclosed herein can be dosed intravenous, subcutaneous, or intramuscular. In certain embodiments, the pharmaceutically acceptable salt of Compound 1, or a crystalline form thereof, disclosed herein are orally bioavailable and can be dosed orally.

In certain embodiments, a pharmaceutically acceptable salt of Compound 1, or a crystalline form thereof, disclosed herein is formulated as a tablet, which may optionally contain one or more other compounds useful for treating HIV. In certain embodiments, the tablet can contain one or more other compounds useful for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, such tablets are suitable for once daily dosing.

### HIV Combination Therapy

In some embodiments, a pharmaceutically acceptable salt of Compound 1, or a crystalline form thereof, disclosed herein is administered with at least one additional therapeutic agent.

In the above embodiments, the additional therapeutic agent may be an anti-HIV agent selected from the group consisting of combination drugs for treating HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, CD4 modulators, CD4 antagonists, HIV ribonuclease H inhibitors, retrocyclin modulators, CDK-9 inhibitors, CCR5 chemokine antagonists, CCR5 gene modulators, dendritic ICAM-3 grabbing nonintegrin 1 inhibitors, HIV GAG protein inhibitors, HIV POL protein inhibitors, hyaluronidase inhibitors, Nef antagonists, Nef inhibitors, Protease-activated receptor-1 antagonists, TNF alpha ligand inhibitors, PDE4 inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, a long-acting HIV regimen, a contraceptive agent, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV nucleoside reverse transcriptase translocation inhibitors, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof. In some embodiments, the additional therapeutic agent is selected from immunomodulators, immunotherapeutic agents, antibody-drug conjugates, gene modifiers, gene editors (such as CRISPR/Cas9, zinc finger nucleases, homing nucleases, synthetic nucleases, TALENs), and cell therapies such as chimeric antigen receptor T-cell, CAR-T (e.g., YESCARTA® (axicabtagene ciloleucel)), and engineered T cell receptors, TCR-T.

### HIV Combination Drugs

Examples of combination drugs include ATRIPLA® (efavirenz, tenofovir disoproxil fumarate, and emtricitabine); BIKTARVY® (bictegravir, emtricitabine, and tenofovir alafenamide); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI™ (efavirenz, lamivudine, and tenofovir disoproxil fumarate); CIMDU™ (lamivudine and tenofovir disoproxil fumarate); tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; cabotegravir and rilpivirine; cabotegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dapivirine and levonorgestrel; dolutegravir+lamivudine; dolutegravir, emtricitabine, and tenofovir alafenamide; elsulfavirine, emtricitabine, and tenofovir disoproxil; lamivudine+abacavir+zidovudine; lamivudine+abacavir; lamivudine+tenofovir disoproxil fumarate; lamivudine+zidovudine+nevirapine; lopinavir+ritonavir; lopinavir+ritonavir+abacavir+lamivudine; lopinavir+ritonavir+zidovudine+lamivudine; tenofovir+lamivudine; tenofovir disoproxil fumarate+emtricitabine+rilpivirine hydrochloride; lopinavir , ritonavir, zidovudine and lamivudine; Vacc-4x and romidepsin; and APH-0812.

### Other HIV Drugs

Examples of other drugs for treating HIV include acemannan, alisporivir, astodrimer, BanLec, CC-11050, deferiprone, Gamimune, griffithsin, metenkefalin, naltrexone, Prolastin, REP 9, RPI-MN, Vorapaxar, VSSP, H1viral, SB-728-T, 1,5-dicaffeoylquinic acid, rHIV7-shl-TAR-CCR5RZ, AAV-eCD4-Ig gene therapy, MazF gene therapy, MK-8527, BlockAide, PSC-RANTES, ABX-464, AG-1105, APH-0812, BIT-225, CYT-107, HGTV-43, HPH-116, HS-10234, IMO-3100, IND-02, MK-1376, MK-2048, MK-4250, MK-8507, MK-8591, NOV-205, PA-1050040 (PA-040), PGN-007, SCY-635, SB-9200, SCB-719, TR-452, TEV-90110, TEV-90112, TEV-90111, TEV-90113, RN-18, Immuglo, and VIR-576.

### HIV Nucleoside Reverse Transcriptase Translocation Inhibitors

Examples of HIV nucleoside reverse transcriptase translocation inhibitors (“NRTTIs”) include 4′-Ethynyl-2-fluoro-2′-deoxyadenosine triphosphate (also known as MK-8591 and EFdA).

### HIV Protease Inhibitors

Examples of HIV protease inhibitors include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, and TMC-310911.

### HIV Reverse Transcriptase Inhibitors

Examples of HIV non-nucleoside or non-nucleotide inhibitors of reverse transcriptase include dapivirine, delavirdine, delavirdine mesylate, doravirine, efavirenz, etravirine, lentinan, MK-8583, nevirapine, rilpivirine, TMC-278LA, ACC-007, AIC-292, KM-023, PC-1005, and elsufavirine (VM-1500). Further examples of non-nucleoside reverse transcriptase inhibitors are disclosed in U.S. Patent Publication No. US2016/0250215.

Examples of HIV nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8591, MK-858, VM-2500, and KP-1461.

In some embodiments, examples of HIV nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, GS-9131, GS-9148, KP-1461, and 4′-ethynyl-2-fluoro-2′-deoxyadenosine (EFdA).

### HIV Integrase Inhibitors

Examples of HIV integrase inhibitors include elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, BMS-986197, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169, VM-3500, and cabotegravir.

Examples of HIV non-catalytic site, or allosteric, integrase inhibitors (NCINI) include CX-05045, CX-05168, and CX-14442.

### HIV Entry Inhibitors

Examples of HIV entry (fusion) inhibitors include cenicriviroc, CCR5 inhibitors, gp41 inhibitors, CD4 attachment inhibitors, DS-003 (BMS-599793), gp120 inhibitors, and CXCR4 inhibitors.

Examples of CCR5 inhibitors include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, and vMIP (Haimipu).

Examples of gp41 inhibitors include albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

Examples of CD4 attachment inhibitors include ibalizumab and CADA analogs

Examples of gp120 inhibitors include Radha-108 (receptol) 3B3-PE38, BanLec, bentonite-based nanomedicine, fostemsavir tromethamine, IQP-0831, and BMS-663068

Examples of CXCR4 inhibitors include plerixafor, ALT-1188, N15 peptide, and vMIP (Haimipu).

### HIV Maturation Inhibitors

Examples of HIV maturation inhibitors include BMS-955176, BMS-986197, GSK-3640254, and GSK-2838232.

### Latency Reversing Agents

Examples of latency reversing agents include histone deacetylase (HDAC) inhibitors, proteasome inhibitors such as velcade and ixazomib citrate, protein kinase C (PKC) activators, Smyd2 inhibitors, BET-bromodomain 4 (BRD4) inhibitors, ionomycin, PMA, SAHA (suberanilohydroxamic acid, or suberoyl, anilide, and hydroxamic acid), IL-15 modulating antibodies, JQ1, disulfiram, amphotericin B, and ubiquitin inhibitors such as largazole analogs, APH-0812, and GSK-343.

Examples of HDAC inhibitors include romidepsin, vorinostat, and panobinostat.

Examples of PKC activators include indolactam, prostratin, ingenol B, and DAG-lactones.

### Capsid Inhibitors

Examples of capsid inhibitors include capsid polymerization inhibitors or capsid disrupting compounds, HIV nucleocapsid p7 (NCp7) inhibitors such as azodicarbonamide, HIV p24 capsid protein inhibitors, GS-6207, AVI-621, AVI-101, AVI-201, AVI-301, and AVI-CAN1-15 series.

### Immune-Based Therapies

Examples of immune-based therapies include toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fc fusion protein, AM-0015, ALT-803, NIZ-985, NKTR-255, NKTR-262, NKTR-214, normferon, peginterferon alfa-2a, peginterferon alfa-2b, recombinant interleukin-15, Xmab-24306, RPI-MN, GS-9620, STING modulators, RIG-I modulators, NOD2 modulators, SB-9200, and IR-103.

Examples of TLR agonists include vesatolimod (GS-9620), GS-986, IR-103, lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod, and RO-7020531.

### Phosphatidylinositol 3-Kinase (PI3K) Inhibitors

Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include PTG-100, TRK-170, abrilumab, etrolizumab, carotegrast methyl, and vedolizumab.

### HIV Antibodies, Bispecific Antibodies, and “Antibody-Like” Therapeutic Proteins

Examples of HIV antibodies, bispecific antibodies, and “antibody-like” therapeutic proteins include DARTs®, DUOBODIES®, BITES®, XmAbs®, TandAbs®, Fab derivatives, bispecific antibodies, trispecific antibodies, multivalent antibodies, bnABs (broadly neutralizing HIV-1 antibodies), BMS-936559, TMB-360, and those targeting HIV gp120 or gp41, antibody-recruiting molecules targeting HIV, anti-CD63 monoclonal antibodies, CD3 bispecific antibodies, CD16 bispecific antibodies, anti-GB virus C antibodies, anti-GP120/CD4, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, Immuglo, MB-66

Examples of those targeting HIV in such a manner include bavituximab, UB-421, C2F5, C2G12, C4E10, C2F5+C2G12+C4E10, 8ANC195, 3-BNC-117, 3BNC117-LS, 3BNC60, D1D2, 10-1074, 10-1074-LS, GS-9722, DH411-2, BG18, PGT145, PGT121, PGT122, PGT-151, PGT-133, PGT-135, PGT-128, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PG9, PG16, 8ANC195, 2Dm2m, 4Dm2m, 6Dm2m, VRC01, VRC-01-LS, PGDM1400, A32, 7B2, 10E8, 10E8VLS, 3810109, 10E8v4, 10E8.4/iMab, VRC-01/PGDM-1400/10E8v4, IMC-HIV, iMabm36, 10E8v4/PGT121-VRC01, eCD4-Ig, IOMA, CAP256-VRC26.25, DRVIA7, SAR-441236,VRC-07-523, VRC07-523LS, VRC-HIVMAB080-00-AB, VRC-HIVMAB060-00-AB, P2G12, and VRC07.

Example of HIV bispecific antibodies include MGD014 and TMB-bispecific.

Example of in vivo delivered bnABs include AAV8-VRCO7 and mRNA encoding anti-HIV antibody VRC01.

### Pharmacokinetic Enhancers

Examples of pharmacokinetic enhancers include cobicistat and ritonavir.

### Additional Therapeutic Agents

Examples of additional therapeutic agents include the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include peptide vaccines, recombinant subunit protein vaccines, live vector vaccines, using viral vectors such as arenavirus, lymphocytic choriomeningitis virus (LCMV), pichinde virus, modified vaccinia Ankara virus (MVA), adenovirus, adeno-associated virus (AAV), vesicular stomatitis virus (VSV) and Chimpanzee adenovirus (ChAd), DNA vaccines, CD4-derived peptide vaccines, vaccine combinations, BG505 SOSIP.664 gp140, rgp120 (AIDSVAX), ALVAC HIV (vCP1521)/AIDSVAX B/E (gp120) (RV144), monomeric gp120 HIV-1 subtype C vaccine, Remune, ITV-1, Contre Vir, Ad4-Env145NFL, Ad5-ENVA-48, HB-500, DCVax-001 (CDX-2401), Vacc-4x, Vacc-05, Vacc-CRX, VVX-004, VAC-3S, multiclade DNA recombinant adenovirus-5 (rAd5), rAd5 gag-pol env A/B/C vaccine, Pennvax-G, Pennvax-GP/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140[delta]V2.TV1+MF-59, rVSVIN HIV-1 gag vaccine, SeV-Gag vaccine, AT-20, DNK-4, ad35-Grin/ENV, TBC-M4, HIVAX, HIVAX-2, NYVAC-HIV-PT1, NYVAC-HIV-PT4, DNA-HIV-PT123, rAAV1-PG9DP, GOVX-B11, GOVX-B21, TVI-HIV-1, Ad-4 (Ad4-env Clade C+Ad4-mGag), Paxvax, EN41-UGR7C, EN41-FPA2, PreVaxTat, AE-H, MYM-V101, CombiHIVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines, gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, recombinant peptide vaccine (HIV infection), NCI, rgp160 HIV vaccine, RNActive HIV vaccine, SCB-703, Tat Oyi vaccine, TBC-M4, therapeutic HIV vaccine, UBI HIV gp120, Vacc-4x+romidepsin, variant gp120 polypeptide vaccine, rAd5 gag-pol env A/B/C vaccine, DNA.HTI, DNA.HTI and MVA.HTI, VRC-HIVDNA016-00-VP+VRC-HIVADV014-00-VP, INO-6145, JNJ-9220, gp145 C.6980; eOD-GT8 60 mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, arenavirus vector-based immunotherapies (Vaxwave, TheraT), MVA-BN HIV-1 vaccine regimen, MVA.tHIVconsv4, MVA.tHIVconsv3, UBI HIV gp120, mRNA based prophylactic vaccines, TBL-1203HI, VRC-HIVRGP096-00-VP, VAX-3S, HIV MAG DNA vaccine.

### Gene Therapy and Cell Therapy

Gene Therapy and Cell Therapy including the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection.

Examples of cell therapy include dendritic cell therapy or T-cell therapy (e.g., CD4-positive T-cells or CD8-positive T-cells).

Examples of a T-cell therapy include a TCR-T cell therapy.

Examples of dendritic cell therapy include AGS-004, C34-CCR5/C34-CXCR4 expressing CD4-positive T cell, and AGT-103-transduced autologous T cell therapy.

Examples of gene editing include CCR5 gene editing drugs (e.g., SB-728T), CCR5 gene inhibitors (e.g., Cal-1), AGT-103-transduced autologous T cell therapy, and AAV-eCD4-Ig gene therapy.

### Gene Editors

Genome editing systems include a CRISPR/Cas9 system, a zinc finger nuclease system, a TALEN system, a homing endonucleases system, and a meganuclease system.

Examples of HIV targeting CRISPR/Cas9 systems include EBT-101.

### CAR-T Cell Therapy

A CAR-T cell therapy can involve a population of immune effector cells engineered to express a chimeric antigen receptor (CAR), wherein the CAR comprises an HIV antigen-binding domain. The HIV antigen can include an HIV envelope protein or a portion thereof, gp120 or a portion thereof, a CD4 binding site on gp120, the CD4-induced binding site on gp120, N-glycan on gp120, the V2 of gp120, the membrane proximal region on gp41. The immune effector cell can be a T-cell or an NK cell. In some embodiments, the T-cell is a CD4-positive T-cell, a CD8-positive T-cell, or a combination thereof. CAR-T cells can be autologous or allogeneic.

Examples of HIV CAR-T include VC-CAR-T, anti-CD4 CART cell therapy, autologous hematopoietic stem cells genetically engineered to express a CD4 CAR and the C46 peptide.

### TCR-T Cell Therapy

TCR-T cells can be engineered to target HIV-derived peptides present on the surface of virus-infected cells.

### Long-Acting HIV Regimen

Examples of drugs in development as long acting regimens include cabotegravir LA, rilpivirine LA, cabotegravir LA+rilpivirine LA, any integrase LA, VM-1500A-LAI, maraviroc (LAI), tenofovir implant, MK-8591 implant, long-acting dolutegravir, long acting raltegravir+lamivudine.

### Contraceptive Agents

Therapeutic agents used for birth control (contraceptive agent) include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### HIV Combination Therapy

In a particular embodiment, a pharmaceutically acceptable salt of Compound 1, or crystalline form thereof, disclosed herein is combined with one, two, three, four or more additional therapeutic agents selected from ATRIPLA® (efavirenz, tenofovir disoproxil fumarate, and emtricitabine); BIKTARVY® (bictegravir, emtricitabine, and tenofovir alafenamide); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI™ (efavirenz, lamivudine, and tenofovir disoproxil fumarate); CIMDU™ (lamivudine and tenofovir disoproxil fumarate); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, a pharmaceutically acceptable salt of Compound 1, or crystalline form thereof, disclosed herein is combined with one, two, three, four or more additional therapeutic agents selected from ATRIPLA® (efavirenz, tenofovir disoproxil fumarate, and emtricitabine); BIKTARVY® (bictegravir, emtricitabine, and tenofovir alafenamide); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI™ (efavirenz, lamivudine, and tenofovir disoproxil fumarate); CIMDU™ (lamivudine and tenofovir disoproxil fumarate); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; abacavir sulfate; 4′-ethynyl-2-fluoro-2′-deoxyadenosine (EFdA); and Bictegravir, or a pharmaceutically acceptable salt thereof.

It will be appreciated by one of skill in the art that the additional therapeutic agents listed above may be included in more than one of the classes listed above. The particular classes are not intended to limit the functionality of those compounds listed in those classes.

In a specific embodiment a pharmaceutically acceptable salt of Compound 1, or a crystalline form thereof, disclosed herein is combined with one or two HIV nucleoside or nucleotide inhibitors of reverse transcriptase. In a specific embodiment, a pharmaceutically acceptable salt of Compound 1, or a crystalline form thereof, disclosed herein is combined with an HIV nucleoside or nucleotide inhibitor of reverse transcriptase and an HIV non-nucleoside inhibitor of reverse transcriptase. In another specific embodiment, a pharmaceutically acceptable salt of Compound 1, or a crystalline form thereof, disclosed herein is combined with an HIV nucleoside or nucleotide inhibitor of reverse transcriptase, and an HIV protease inhibiting compound. In an additional embodiment, a pharmaceutically acceptable salt of Compound 1, or a crystalline form thereof, disclosed herein is combined with an HIV nucleoside or nucleotide inhibitor of reverse transcriptase, an HIV non-nucleoside inhibitor of reverse transcriptase, and a pharmacokinetic enhancer. In certain embodiments, a pharmaceutically acceptable salt of Compound 1, or a crystalline form thereof, disclosed herein is combined with at least one HIV nucleoside inhibitor of reverse transcriptase, an integrase inhibitor, and a pharmacokinetic enhancer. In another embodiment, a pharmaceutically acceptable salt of Compound 1, or a crystalline form thereof, disclosed herein is combined with two HIV nucleoside or nucleotide inhibitors of reverse transcriptase.

In some embodiments, a pharmaceutically acceptable salt of Compound 1, or a crystalline form thereof, disclosed herein is combined with one or two HIV nucleoside or nucleotide inhibitors of reverse transcriptase. In a specific embodiment, a pharmaceutically acceptable salt of Compound 1, or a crystalline form thereof, disclosed herein is combined with an HIV nucleoside or nucleotide inhibitor of reverse transcriptase and an HIV non-nucleoside inhibitor of reverse transcriptase. In another specific embodiment, a pharmaceutically acceptable salt of Compound 1, or a crystalline form thereof, disclosed herein is combined with an HIV nucleoside or nucleotide inhibitor of reverse transcriptase, and an HIV protease inhibiting compound. In an additional embodiment, a pharmaceutically acceptable salt of Compound 1, or a crystalline form thereof, disclosed herein is combined with an HIV nucleoside or nucleotide inhibitor of reverse transcriptase, an HIV non-nucleoside inhibitor of reverse transcriptase, and a pharmacokinetic enhancer. In certain embodiments, a pharmaceutically acceptable salt of Compound 1, or a crystalline form thereof, disclosed herein is combined with at least one HIV nucleoside inhibitor of reverse transcriptase, an integrase inhibitor, and a pharmacokinetic enhancer. In another embodiment, a pharmaceutically acceptable salt of Compound 1, or a crystalline form thereof, disclosed herein is combined with two HIV nucleoside or nucleotide inhibitors of reverse transcriptase.

In a particular embodiment, a pharmaceutically acceptable salt of Compound 1, or a crystalline form thereof, disclosed herein is combined with abacavir sulfate, tenofovir, tenofovir disoproxil, tenofovir disoproxil fumarate, tenofovir disoproxil hemifumarate, tenofovir alafenamide, tenofovir alafenamide fumarate or tenofovir alafenamide hemifumarate.

In some embodiments, a pharmaceutically acceptable salt of Compound 1, or a crystalline form thereof, disclosed herein is combined with abacavir sulfate, tenofovir, tenofovir disoproxil, tenofovir disoproxil fumarate, tenofovir disoproxil hemifumarate, tenofovir alafenamide, tenofovir alafenamide fumarate, tenofovir alafenamide hemifumarate, bictegravir (or a pharmaceutically acceptable salt thereof), or 4′-ethynyl-2-fluoro-2′-deoxyadenosine (EFdA).

In a particular embodiment, a pharmaceutically acceptable salt of Compound 1, or a crystalline form thereof, disclosed herein is combined with tenofovir, tenofovir disoproxil, tenofovir disoproxil fumarate, tenofovir alafenamide, tenofovir alafenamide fumarate or tenofovir alafenamide hemifumarate.

In some embodiments, a pharmaceutically acceptable salt of Compound 1, or a crystalline form thereof, disclosed herein is combined with tenofovir, tenofovir disoproxil, tenofovir disoproxil fumarate, tenofovir alafenamide, tenofovir alafenamide fumarate, tenofovir alafenamide hemifumarate, bictegravir (or a pharmaceutically acceptable salt thereof), or 4′-ethynyl-2-fluoro-2′-deoxyadenosine (EFdA).

In some embodiments, a pharmaceutically acceptable salt of Compound 1, or a crystalline form thereof, disclosed herein a pharmaceutically acceptable salt of Compound 1, or crystalline form thereof, disclosed herein is combined with a first additional therapeutic agent selected from the group consisting of abacavir sulfate, tenofovir, tenofovir disoproxil, tenofovir disoproxil fumarate, tenofovir alafenamide, tenofovir alafenamide fumarate and tenofovir alafenamide hemifumarate, and a second additional therapeutic agent selected from the group consisting of emtricitabine and lamivudine.

In a particular embodiment, a pharmaceutically acceptable salt of Compound 1, or a crystalline form thereof, disclosed herein is combined with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine. In a particular embodiment, a pharmaceutically acceptable salt of Compound 1, or a crystalline form thereof, disclosed herein is combined with a first additional therapeutic agent selected from the group consisting of tenofovir alafenamide fumarate, tenofovir alafenamide, and tenofovir alafenamide hemifumarate, and a second additional therapeutic agent, wherein the second additional therapeutic agent is emtricitabine. In a particular embodiment, a pharmaceutically acceptable salt of Compound 1, or a crystalline form thereof, disclosed herein is combined with a first additional therapeutic agent selected from the group consisting of tenofovir disoproxil fumarate, tenofovir disoproxil, and tenofovir disoproxil hemifumarate, and a second additional therapeutic agent, wherein the second additional therapeutic agent is emtricitabine. In some embodiments, the pharmaceutically acceptable salt of Compound 1, or a crystalline form thereof, and the first and second additional therapeutic agents as disclosed above are administered simultaneously. Optionally, the pharmaceutically acceptable salt of Compound 1, or a crystalline form thereof, and the first and second additional therapeutic agents as disclosed above are combined in a unitary dosage form for simultaneous administration to a subject. In other embodiments, the pharmaceutically acceptable salt of Compound 1, or a crystalline form thereof, and the first and second additional therapeutic agents as disclosed above are administered sequentially.

In some embodiments, a pharmaceutically acceptable salt of Compound 1, or a crystalline form thereof, disclosed herein is combined with bictegravir or a pharmaceutically acceptable salt thereof.

In some embodiments, a pharmaceutically acceptable salt of Compound 1, or a crystalline form thereof, disclosed herein is combined with 4′-ethynyl-2-fluoro-2′-deoxyadenosine (EFdA).

A pharmaceutically acceptable salt of Compound 1, or a crystalline form thereof, disclosed herein may be combined with one or more additional therapeutic agents in any dosage amount of the pharmaceutically acceptable salt of Compound 1, or a crystalline form thereof (e.g., from 1 mg to 1000 mg of the salt or crystalline form).

In some embodiments, a pharmaceutically acceptable salt of Compound 1, or a crystalline form thereof, disclosed herein may be combined with one or more additional therapeutic agents in any dosage amount of the pharmaceutically acceptable salt of Compound 1, or a crystalline form thereof (e.g., from 1 mg to 1000 mg of the salt or crystalline form).

In certain embodiments, a pharmaceutically acceptable salt of Compound 1, or a crystalline form thereof, disclosed herein is combined with 5-30 mg tenofovir alafenamide fumarate, tenofovir alafenamide hemifumarate, or tenofovir alafenamide, and 200 mg emtricitabine. In certain embodiments, a pharmaceutically acceptable salt of Compound 1, or a crystalline form thereof, disclosed herein is combined with 5-10, 5-15, 5-20, 5-25, 25-30, 20-30, 15-30, or 10-30 mg tenofovir alafenamide fumarate, tenofovir alafenamide hemifumarate, or tenofovir alafenamide, and 200 mg emtricitabine. In certain embodiments, a pharmaceutically acceptable salt of Compound 1, or a crystalline form thereof, disclosed herein is combined with 10 mg tenofovir alafenamide fumarate, tenofovir alafenamide hemifumarate, or tenofovir alafenamide, and 200 mg emtricitabine. In certain embodiments, a pharmaceutically acceptable salt of Compound 1, or a crystalline form thereof, disclosed herein is combined with 25 mg tenofovir alafenamide fumarate, tenofovir alafenamide hemifumarate, or tenofovir alafenamide, and 200 mg emtricitabine. A pharmaceutically acceptable salt of Compound 1, or a crystalline form thereof, disclosed herein may be combined with the agents provided herein in any dosage amount of the salt or crystalline form (e.g., from 1 mg to 1000 mg of the salt or crystalline form) the same as if each combination of dosages were specifically and individually listed.

In certain embodiments, a pharmaceutically acceptable salt of Compound 1, or a crystalline form thereof, disclosed herein, is combined with 200-400 mg tenofovir disoproxil fumarate, tenofovir disoproxil hemifumarate, or tenofovir disoproxil, and 200 mg emtricitabine. In certain embodiments, a pharmaceutically acceptable salt of Compound 1, or a crystalline form thereof, disclosed herein is combined with 200-250, 200-300, 200-350, 250-350, 250-400, 350-400, 300-400, or 250-400 mg tenofovir disoproxil fumarate, tenofovir disoproxil hemifumarate, or tenofovir disoproxil, and 200 mg emtricitabine. In certain embodiments, a pharmaceutically acceptable salt of Compound 1, or a crystalline form thereof, disclosed herein is combined with 300 mg tenofovir disoproxil fumarate, tenofovir disoproxil hemifumarate, or tenofovir disoproxil, and 200 mg emtricitabine. A pharmaceutically acceptable salt of Compound 1, or a crystalline form thereof, disclosed herein may be combined with the agents provided herein in any dosage amount of the salt or crystalline form (e.g., from 1 mg to 1000 mg of the salt or crystalline form) the same as if each combination of dosages were specifically and individually listed.

In some embodiments, a pharmaceutically acceptable salt of Compound 1, or a crystalline form thereof, disclosed herein is combined with 20-80 mg of bictegravir or a pharmaceutically acceptable salt thereof. A pharmaceutically acceptable salt of Compound 1, or a crystalline form thereof, disclosed herein may be combined with the agents provided herein in any dosage amount of the salt or crystalline form (e.g., from 1 mg to 1000 mg of the salt or crystalline form) the same as if each combination of dosages were specifically and individually listed.

In one embodiment, kits comprising a pharmaceutically acceptable salt of Compound 1, or a crystalline form thereof, disclosed herein in combination with one or more (e.g., one, two, three, one or two, or one to three) additional therapeutic agents are provided.

In some embodiments, kits comprising a pharmaceutically acceptable salt of Compound 1, or a crystalline form thereof, disclosed herein in combination with one or more (e.g., one, two, three, one or two, or one to three) additional therapeutic agents are provided.

### Pharmaceutical Compositions

Pharmaceutical compositions disclosed herein comprise a pharmaceutically acceptable salt of Compound 1, or a crystalline form thereof, disclosed herein together with one or more pharmaceutically acceptable excipients and optionally other therapeutic agents. Pharmaceutical compositions containing the active ingredient may be in any form suitable for the intended method of administration.

In some embodiments, pharmaceutical compositions disclosed herein comprise a pharmaceutically acceptable salt of Compound 1, or a crystalline form thereof, disclosed herein together with one or more pharmaceutically acceptable excipients and optionally other therapeutic agents. Pharmaceutical compositions containing the active ingredient may be in any form suitable for the intended method of administration.

Pharmaceutical compositions comprising the pharmaceutically acceptable salt of Compound 1, or a crystalline form thereof, disclosed herein may be prepared with conventional carriers (e.g., inactive ingredient or excipient material) which may be selected in accord with ordinary practice. Tablets may contain excipients including glidants, fillers, binders and the like. Aqueous compositions may be prepared in sterile form, and when intended for delivery by other than oral administration generally may be isotonic. All compositions may optionally contain excipients such as those set forth in the Rowe et al, Handbook of Pharmaceutical Excipients, 5th edition, American Pharmacists Association, 1986. Excipients can include ascorbic acid and other antioxidants, chelating agents such as EDTA, carbohydrates such as dextrin, hydroxyalkylcellulose, hydroxyalkylmethylcellulose, stearic acid and the like.

While it is possible for the active ingredient to be administered alone, it may be preferable to present the active ingredient as pharmaceutical compositions. The compositions, both for veterinary and for human use, comprise at least the pharmaceutically acceptable salt of Compound 1, or a crystalline form thereof, disclosed herein together with one or more acceptable carriers and optionally other therapeutic ingredients. In one embodiment, the pharmaceutical composition comprises a pharmaceutically acceptable salt of Compound 1, or a crystalline form thereof, a pharmaceutically acceptable excipient, and a therapeutically effective amount of one or more (e.g., one, two, three, or four; or one or two; or one to three; or one to four) additional therapeutic agents as defined hereinbefore. In one embodiment, the pharmaceutical composition comprises a pharmaceutically acceptable salt of Compound 1, or a crystalline form thereof, a pharmaceutically acceptable excipient, and one other therapeutic ingredient. The carrier(s) are “acceptable” in the sense of being compatible with the other ingredients of the composition and physiologically innocuous to the recipient thereof.

The compositions include those suitable for various administration routes. The compositions may conveniently be presented in unit dosage form and may be prepared by any of the methods well known in the art of pharmacy. Such methods include the step of bringing into association the active ingredient with one or more inactive ingredients (e.g., a carrier, pharmaceutical excipient, etc.). The compositions may be prepared by uniformly and intimately bringing into association the active ingredient with liquid carriers or finely divided solid carriers or both, and then, if necessary, shaping the product. Techniques and formulations generally are found in Remington: The Science and Practice of Pharmacy, 21^{st} Edition, Lippincott Wiliams and Wilkins, Philadelphia, Pa., 2006.

Compositions described herein that are suitable for oral administration may be presented as discrete units (a unit dosage form) including but not limited to capsules, cachets or tablets each containing a predetermined amount of the active ingredient.

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, disclosed herein are oral dosage forms (e.g., tablets), which may be prepared from hot melt extrusion or spray-drying dispersion (SDD) technologies.

In some embodiments, disclosed herein are hard capsules filled with powder, beads, or granules containing the active ingredient in admixture with non-toxic pharmaceutically acceptable excipient which are suitable for manufacture of hard or soft capsules. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc.

In some embodiments, disclosed herein are hard or soft capsules filled with liquid or semi-solid mixtures containing the active ingredient in admixture with non-toxic pharmaceutically acceptable excipient which are suitable for manufacture of hard or soft capsules. These excipients may be, for example, solubilizing oils such as maize oil, sesame oil, or corn oil; medium chain triglycerides and related esters, such as, derivitized palm kernel oil or coconut oil; self-emulsifying lipid systems (SEDDS or SMEDDS), such as caprylic triglyceride or propylene glycol monocaprylate; viscosity modifiers, such as, cetyl alcohol, steryl alcohol, glycerol stearate; and solubilizing agents and surfactants, such as polyethylene glycol, propylene glycol, glycerin, ethanol, polyethoxylated castor oil, poloxamers, or polysorbates.

The pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the sterile injectable preparation disclosed herein may also be a sterile injectable solution or suspension prepared from a reconstituted lyophilized powder in a non-toxic parenterally acceptable diluent or solvent, such as a solution in 1,3-butane-diol. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In certain embodiments the suspension is a microsuspension. In certain embodiments the suspension is a nanosuspension.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and may be found e.g., in Handbook of Pharmaceutical Excipients (eds. Rowe, Sheskey & Quinn), 6th edition 2009.

Examples of solubilizing excipients in a parenteral formulation (e.g., an SC or IM formulation) include, but are not limited to, polysorbates (such as polysorbate 20 or 80) and poloxamers (such as poloxamer 338, 188, or 207). In some embodiments, disclosed herein is a parenteral administration (e.g., an SC or IM formulation) that comprises a pharmaceutically acceptable salt of Compound 1, or crystalline form thereof, disclosed herein and a poloxamer, in particular poloxamer 338. In some embodiments, the amount of poloxamer (e.g., poloxamer 388) in a parenteral administration disclosed herein is less than about 5%, such as less than about 3%, about 2%, about 1%, or about 0.5%.

In some embodiments, the parenteral formulation (e.g., an SC or IM formulation) disclosed herein is an aqueous suspension. In some embodiments, the parenteral formulation (e.g., an SC or IM formulation) disclosed herein is an aqueous suspension that comprises a pharmaceutically acceptable salt of Compound 1, or a crystalline form thereof, disclosed herein and saline. In some embodiments, the parenteral formulation (e.g., an SC or IM formulation) disclosed herein is an aqueous suspension that comprises a pharmaceutically acceptable salt of Compound 1, or a crystalline form thereof, disclosed herein saline, and a poloxamer (such as poloxamer 338, 188, or 207).

In certain embodiments, the composition is disclosed as a solid dosage form, including a solid injectable dosage form, such as a solid depot form.

The amount of active ingredient that may be combined with the inactive ingredients to produce a dosage form may vary depending upon the intended treatment subject and the particular mode of administration. For example, in some embodiments, a dosage form for oral administration to humans may contain approximately 1 to 1000 mg of active material formulated with an appropriate and convenient amount of carrier material (e.g., inactive ingredient or excipient material). In certain embodiments, the carrier material varies from about 5 to about 95% of the total compositions (weight:weight).

It should be understood that in addition to the ingredients particularly mentioned above the compositions of these embodiments may include other agents conventional in the art having regard to the type of composition in question, for example those suitable for oral administration may include flavoring agents.

In certain embodiments, a composition comprising an active ingredient disclosed herein in one variation does not contain an agent that affects the rate at which the active ingredient is metabolized. Thus, it is understood that compositions comprising a pharmaceutically acceptable salt of Compound 1, or a crystalline form thereof, in certain embodiments do not comprise an agent that would affect (e.g., slow, hinder or retard) the metabolism of the pharmaceutically acceptable salt of Compound 1, or a crystalline form thereof, or any other active ingredient administered separately, sequentially or simultaneously with the salt or crystalline form. It is also understood that any of the methods, kits, articles of manufacture and the like detailed herein in certain embodiments do not comprise an agent that would affect (e.g., slow, hinder or retard) the metabolism of a pharmaceutically acceptable salt of Compound 1, or a crystalline form thereof, or any other active ingredient administered separately, sequentially or simultaneously with the pharmaceutically acceptable salt of Compound 1, or a crystalline form thereof.

### Kits and Articles of Manufacture

The present disclosure relates to a kit comprising a pharmaceutically acceptable salt of Compound 1, or a crystalline form thereof, disclosed herein. In one embodiment, the kit may comprise one or more additional therapeutic agents as described hereinbefore. The kit may further comprise instructions for use, e.g., for use in inhibiting an HIV reverse transcriptase, such as for use in treating an HIV infection or AIDS or as a research tool. The instructions for use are generally written instructions, although electronic storage media (e.g., magnetic diskette or optical disk) containing instructions are also acceptable.

The present disclosure also relates to a pharmaceutical kit comprising one or more containers comprising a pharmaceutically acceptable salt of Compound 1, or a crystalline form thereof, disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

In some embodiments, the present disclosure also relates to a pharmaceutical kit comprising one or more containers comprising a pharmaceutically acceptable salt of Compound 1, or a crystalline form thereof, disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

Also disclosed are articles of manufacture comprising a unit dosage of a pharmaceutically acceptable salt of Compound 1, or a crystalline form thereof, disclosed herein in suitable packaging for use in the methods described herein. Suitable packaging is known in the art and includes, for example, vials, vessels, ampules, bottles, jars, flexible packaging and the like. An article of manufacture may further be sterilized and/or sealed.

### EXAMPLES

### General Methods

### X-Ray Powder Diffraction (XRPD)

XRPD patterns were collected on a PANanalytical XPERT-PRO diffractometer using CuK_{α1} radiation at ambient conditions under the following experimental settings: 45 KV, 40 mA, Kα1=1.5406 Å, scan range 2 to 40°, step size 0.0084 or 0.0167°, measurement time: 5 min.

Differential Scanning calorimetry (DSC)

DSC thermograms were collected on a TA Instruments Q2000 system equipped with a 50-position auto-sampler. The calibration for energy and temperature was carried out using certified indium. Typically, 1-5 mg of each sample, in a pin-holed aluminium pan, was heated at 10° C./min from 25° C. to 300° C. A purge of dry nitrogen at 50 mL/min was maintained over the sample throughout the measurement. The onset of the melting endotherm was reported as the melting point.

### Thermo-Gravimetric Analysis (TGA)

TGA thermograms were collected on a TA Instruments Q5000 system, equipped with a 25-position auto-sampler. Typically, 1-5 mg of each sample was loaded onto a pre-tared aluminium pan and heated at 10° C./min from 25° C. to 250° C. A nitrogen purge at 25 mL/min was maintained over the sample throughout the measurement.

### Dynamic Vapor Sorption (DVS)

DVS data, which was used to determine the hygroscopicity of solids, were collected on a TA Instruments Q5000SA system. The temperature-controlled chamber was set at 25° C. and dry nitrogen was introduced at a flow rate of 10 mL/min. Approximately 1 to 5 mg sample was placed in a semispherical metal-coated quartz crucible or a disposable aluminum pan. A stepwise isotherm experiment at 25° C. was conducted by controlling the relative humidity (RH) in the chamber from 10% to 90%, then down to 10%, at 10% increments to accomplish a full sorption/desorption cycle.

### Proton Nuclear Magnetic Resonance CH NMR)

¹H NMR spectra were collected on a Varian 400-MR 400MHz instrument with 7620AS sample changer. The default proton parameters are as follows: spectral width: 14 to −2 ppm (6397.4 Hz); relaxation delay: 1 sec; pulse: 45 degrees; acquisition time: 2.049 sec; number of scans or repetitions: 8; temperature: 25° C. Samples were prepared in dimethyl sulfoxide-d6, unless otherwise stated. Off-line analysis was carried out using MestReNova software.

### Preparation of (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl (1-(difluoromethyl)cyclopropyl)carbamate Amorphous Free Base

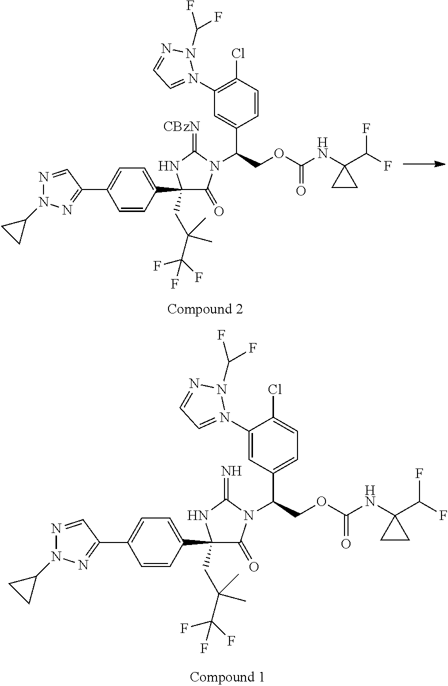

(S)-2-((R)-2-(((benzyloxy)carbonyl)5 imino)-4-(4-(2-cyclopropyl-2H-1,2,3-triazol-4-yl)phenyl)-5-oxo-4-(3,3,3-trifluoro-2,2-dimethylpropyl)imidazolidin-1-yl)-2-(4-chloro-3-(1-(difluoromethyl)-1H-1,2,4-triazol-5-yl)phenyl)ethyl (1-(difluoromethyl)cyclopropyl)carbamate (Compound 2) was prepared as described in U.S. patent application Ser. No. 16/158,446, filed Oct. 12, 2018, the entire contents of which are incorporated herein by reference in their entireties. Compound 2 (70.7 mmol) was dissolved in TFA (267 mL) and heated at 70° C. for 3 hr. After cooling to room temperature, the mixture was concentrated to dryness followed by co-evaporation with toluene (2 x 100 mL). The residue was taken up in isopropyl acetate (335 mL) and neutralized with saturated aqueous sodium bicarbonate (200 mL) followed by the addition of 1.0 M aqueous sodium hydroxide (335 mL). The solvent layers were separated and the organic layer was washed with 10 wt % aqueous sodium chloride. Resulting solvent layers were separated and the organic layer was concentrated. The residue was purified by column chromatography on silica gel eluting with 0-70% EtOAc in hexanes to afford the product.

### Example 1. Polymorph Screening of (S)-2-(4-chloro-3-(1-(difluoromethyl)-1H-1,2,4-triazol-5-yl)phenyl)-2-4R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl(1-(difluoromethyl)cyclopropyl)carbamate Hydrochloride Salt

Amorphous Compound 1 hydrochloride salt was prepared as follows: Compound 1 free base was mixed with 1 equivalent of concentrated HC1 solution in 20 vol acetonitrile to obtain a solution, which was subsequently dried at 50° C. to become a foam.

Polymorph screening was conducted by stirring amorphous Compound 1 hydrochloride salt in various solvents at 22° C. and 0 to 5° C. No unsolvated form of Compound 1 hydrochloride salt was obtained. Some solvents produced solvate forms of Compound 1 hydrochloride salt, as shown in Table 1. Most solvates formed amorphous or highly disordered solids after full desolvation (Table 1, Dry form). The solvate from CPME became crystalline Form I, the solvate from DCM became crystalline Form II; and the solvate from EtOAc became crystalline Form III.

TABLE 1

| Solvent | Antisolvent | Wet form | Dry form |
|---|---|---|---|
| IPA | heptanes | IPA Solvate | Amorphous |
| 2-butanol | IPE | 2-butanol solvate | Form I + amorphous |
| t-butanol | IPE | t-butanol solvate | Amorphous |
| THF | heptanes | THF solvate | Amorphous |
| THF | MTBE | THF/MTBE | Form I + amorphous |
| | | co-solvate | |
| 2-MeTHF | heptanes | 2-MeTHF solvate | Form I + amorphous |
| CPME | heptanes | CPME solvate | Form I |
| MTBE | NA | MTBE solvate | Material A |
| Ethyl ether | NA | Ethyl ether solvate | Material A |
| Anisole | NA | Anisole solvate | Amorphous |
| Ethyl formate | NA | Ethyl formate solvate | Material B |
| EtOAc | heptanes | EtOAc solvate | Form III |
| n-BuOAc | NA | n-BuOAc solvate | Material B |
| DCM | heptanes | DCM solvate | Form II |

NA refers to no data available.

### THF Solvate

The THF solvate form of Compound 1 hydrochloride salt was first crystallized after stirring amorphous Compound 1 hydrochloride salt in a mixture of THF and heptanes at 0° C. to 5° C. for about 16 hours.

The THF solvate was reproduced by slurrying amorphous Compound 1 hydrochloride salt in 5 vol THF, seeding with crystallized THF solvate, adding 2 vol heptanes, and stirring at about 22° C. The solids were isolated by filtration and drying at 50° C. under vacuum.

The XRPD pattern of the THF solvate form of Compound 1 hydrochloride salt is shown in FIG. 1.

### Form I

As shown in Table 1, Compound 1 hydrochloride salt, crystalline Form I was obtained after desolvating the CPME. Form I of diminished crystallinity was obtained by desolvating other solvate forms of Compound 1 hydrochloride salt, such as THF/MTBE co-solvates, 2-BuOH solvates, 2-MeTHF solvates and IPA solvates.

XRPD patterns of the hydrochloride salt of Compound 1, crystalline Form I obtained after desolvating CPME solvate, THF/MTBE co-solvate, 2-BuOH solvate, 2-MeTHF solvate, and IPA solvate are shown in FIG. 2 (top to bottom).

Compound 1 hydrochloride salt, crystalline Form I was first prepared as follows: A mixture of 50 mg Compound 1 hydrochloride salt, 50 μL of CPME and 10 μL heptanes were stirred at 0° C. to 5° C. for 15 hours, and remained as a solution. About 1 mg of seeds of the THF solvate form of Compound 1 hydrochloride salt was added, and the solution crystallized. The resulting solids were filtered and wet solids were analyzed by XRPD. The wet cake was dried in a vacuum oven at 90° C. Dried solids showed different XRPD patterns from wet solids. The XRPD pattern from dry solids is characteristic of crystalline Form I of Compound 1 hydrochloride salt.

The XRPD pattern of Compound 1 hydrochloride salt, crystalline Form I is shown in FIG. 3. A list of 2-theta peaks is provided in Table 2.

TABLE 2

| No. | Pos. [°2Th.] | Rel. Int. [%] |
|---|---|---|
| 1 | 6.2 | 10 |
| 2 | 10.4 | 20 |
| 3 | 11.3 | 16 |
| 4 | 12.6 | 39 |
| 5 | 13.7 | 46 |
| 6 | 14.3 | 8 |
| 7 | 15.1 | 51 |
| 8 | 15.5 | 53 |
| 9 | 16.3 | 74 |
| 10 | 17.6 | 63 |
| 11 | 17.8 | 91 |
| 12 | 18.5 | 25 |
| 13 | 19.6 | 40 |
| 14 | 20.3 | 46 |
| 15 | 21.1 | 22 |
| 16 | 21.7 | 98 |
| 17 | 22.2 | 100 |
| 18 | 23.1 | 19 |
| 19 | 24.6 | 65 |
| 20 | 25.3 | 46 |
| 21 | 26.0 | 42 |
| 22 | 26.8 | 48 |
| 23 | 27.8 | 19 |
| 24 | 29.7 | 8 |
| 25 | 31.2 | 15 |
| 26 | 32.8 | 14 |
| 27 | 36.1 | 6 |
| 28 | 37.5 | 12 |

The TGA thermogram of Compound 1 hydrochloride salt, crystalline Form I is shown in FIG. 4. A weight loss of about 2% from 25° C. to 150° C. was observed.

The DSC thermogram of Compound 1 hydrochloride salt, crystalline Form I is shown in FIG. 5. Two endothermic events were observed: one with onset at about 98° C. is believed to be due to solvent loss, and a second one with onset at about 140° C. due to melting.

The DVS sorption isotherm of Compound 1, hydrochloride salt, crystalline Form I is shown in FIG. 6. The solids were found to be slightly hygroscopic, as indicated by 1.8% water uptake over the entire range of 10% to 90% RH at 25° C.

### DCM Solvate

The DCM solvate of Compound 1 hydrochloride salt was first crystallized after stirring a solution of Compound 1 hydrochloride salt in DCM at 22° C. for 10 days.

DCM solvate was reproduced in a 3 g batch as follows: Compound 1 hydrochloride salt was dissolved in 3 vol DCM, polish filtered, charged with 2 vol heptanes, seeded with 0.5% of DCM solvate described above and stirred at about 22° C. A thin slurry formed after stirring overnight. To the slurry, 1 vol of heptanes, followed by 1 vol of n-butyl ether (BE), and 0.3 vol DCM were charged. The resulting solids were isolated by filtration.

The XRPD pattern of the DCM solvate of Compound 1 hydrochloride salt is shown in FIG. 7.

### Form II

Compound 1 hydrochloride salt, crystalline Form II was obtained after desolvating the DCM solvate form of Compound 1 hydrochloride salt under vacuum or at elevated temperatures (e.g., about 50° C.).

The XRPD pattern of Compound 1 hydrochloride salt, crystalline Form II is shown in FIG. 8. A list of 2-theta peaks is provided in Table 3 below.

TABLE 3

| No. | Pos. [°2Th.] | Rel. Int. [%] |
|---|---|---|
| 1 | 6.5 | 14 |
| 2 | 7.2 | 13 |
| 3 | 8.5 | 11 |
| 4 | 9.7 | 4 |
| 5 | 10.3 | 7 |
| 6 | 11.3 | 13 |
| 7 | 11.8 | 6 |
| 8 | 12.5 | 13 |
| 9 | 13.4 | 21 |
| 10 | 14.3 | 100 |
| 11 | 15.7 | 29 |
| 12 | 16.0 | 16 |
| 13 | 17.0 | 51 |
| 14 | 17.7 | 35 |
| 15 | 18.1 | 54 |
| 16 | 18.4 | 28 |
| 17 | 19.4 | 33 |
| 18 | 20.0 | 30 |
| 19 | 20.8 | 28 |
| 20 | 21.4 | 26 |
| 21 | 22.1 | 16 |
| 22 | 23.0 | 19 |
| 23 | 23.8 | 8 |
| 24 | 25.7 | 11 |
| 25 | 26.5 | 7 |
| 26 | 28.3 | 7 |
| 27 | 29.4 | 7 |
| 28 | 30.3 | 6 |
| 29 | 31.1 | 7 |
| 30 | 32.5 | 5 |
| 31 | 34.7 | 7 |
| 32 | 38.0 | 6 |

The TGA thermogram of Compound 1 hydrochloride salt, crystalline Form II is shown in FIG. 9. A first weight loss of about 1% was observed from 25° C. to 70° C., and a second weight loss of about 0.4% from 70° C. to 150° C.

Proton NMR showed 0.7% residual DCM in crystalline Form II solids of Compound 1 hydrochloride salt (data not shown).

The DSC thermogram of Compound 1 hydrochloride salt, crystalline Form II is shown in FIG. 10, indicating a melting onset at about 141° C.

The DVS sorption isotherm of Compound 1 hydrochloride salt, crystalline Form II is shown on FIG. 11. Crystalline Form II was found to be moderately hygroscopic, as indicated by about 3% water uptake from 0% to 90% RH at 25° C.

### EtOAc Solvate

The EtOAc solvate of Compound 1 hydrochloride salt was first crystallized after stirring a solution of Compound 1 hydrochloride in EtOAc at 22° C. in the presence of seeds of Compound 1 hydrochloride salt, crystalline Form II.

The XRPD pattern of the EtOAc solvate of Compound 1 hydrochloride salt is shown in FIG. 12.

Single crystals of the EtOAc solvate were prepared by dissolving 50 mg Compound I hydrochloride salt in 200 μL of EtOAc, charging 100 μL of heptane, seeding with Compound 1 hydrochloride EtOAc solvate, and growing at about 22° C. for three days. The sample was analyzed by SCXRD and results are shown in FIG. 13 and Table 4.

TABLE 4

| Temperature (K) | 100(2) | |
|---|---|---|
| Crystal system | Triclinic | |
| Space group | P 1 | |
| Unit cell parameters | a = 11.65967(19) Å | α = 79.8278(19)° |
| | b = 13.6291(3) Å | β = 68.0006(18)° |
| | c = 15.3692(3) Å | γ = 78.9173(17)° |

| | |
|---|---|
| Calculated density (g/cm³) | 1.408 |

### Form III

As shown in Table 1, Compound 1 hydrochloride salt, crystalline Form III was obtained by first forming solvates of the hydrochloride salt of Compound 1 in EtOAc, followed by desolvation at about 50° C. or above under vacuum.

The XRPD pattern of Compound I hydrochloride salt, crystalline Form III is shown in FIG. 14. A list of 2-theta peaks is provided in Table 5 below.

TABLE 5

| No. | Pos. [°2Th.] | Rel. Int. [%] |
|---|---|---|
| 1 | 6.3 | 25 |
| 2 | 6.6 | 27 |
| 3 | 7.7 | 12 |
| 4 | 8.7 | 14 |
| 5 | 9.0 | 9 |
| 6 | 9.7 | 17 |
| 7 | 10.9 | 55 |
| 8 | 11.4 | 5 |
| 9 | 12.3 | 16 |
| 10 | 12.9 | 22 |
| 11 | 13.7 | 40 |
| 12 | 14.1 | 13 |
| 13 | 15.3 | 83 |
| 14 | 16.0 | 37 |
| 15 | 17.0 | 51 |
| 16 | 17.3 | 61 |
| 17 | 17.8 | 57 |
| 18 | 18.5 | 100 |
| 19 | 19.7 | 58 |
| 20 | 20.0 | 82 |
| 21 | 20.5 | 33 |
| 22 | 21.6 | 52 |
| 23 | 21.8 | 44 |
| 24 | 22.4 | 39 |
| 25 | 23.0 | 49 |
| 26 | 23.7 | 26 |
| 27 | 24.4 | 29 |
| 28 | 25.1 | 47 |
| 29 | 25.8 | 19 |
| 30 | 27.3 | 34 |
| 31 | 28.1 | 39 |
| 32 | 28.5 | 30 |
| 33 | 29.4 | 17 |
| 34 | 30.4 | 22 |
| 35 | 32.8 | 11 |
| 36 | 34.4 | 10 |
| 37 | 37.6 | 7 |

The TGA thermogram of Compound I hydrochloride salt, crystalline Form III is shown in FIG. 15. A weight loss of about 0.4% from 25° C. to 150° C. was observed.

The DSC thermogram of Compound 1 hydrochloride salt, crystalline Form III is shown in FIG. 16, exhibiting a melting onset of about 145° C.

The DVS sorption isotherm of Compound 1 hydrochloride salt, crystalline Form III is shown in FIG. 17. Crystalline Form III was slightly hygroscopic, as indicated by about 1.4% water uptake over the entire range of 0% to 90% RH at 25° C.

### Material A

As shown in Table 1, Compound 1 hydrochloride salt, Material A was obtained after desolvating the ethyl ether solvate or MTBE solvate of Compound 1 hydrochloride salt. The XRPD pattern of Compound 1 hydrochloride salt, Material A is shown in FIG. 18.

### Material B

As shown in Table 1, Compound 1 hydrochloride salt, Material B was obtained after desolvating the ethyl formate or butyl acetate solvate of Compound 1 hydrochloride salt. The XRPD pattern of Compound 1 hydrochloride salt, Material B is shown in FIG. 19.

### Example 2. Polymorph Screening of (S)-2-(4-chloro-3-(1-(difluoromethyl)-1H-1,2,4-triazol-5-yl)phenyl)-2-4R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl(1-(difluoromethyl)cyclopropyl)carbamate Phosphate Salt

Amorphous Compound 1 phosphate salt was prepared as follows: Compound 1 free base was mixed with 1 equivalent of phosphoric acid (85% aqueous solution) in 5 vol MTBE to obtain a solution, which was subsequently dried at 50° C. to become a foam.

A polymorph screening of Compound 1 phosphate salt was conducted by stirring amorphous Compound 1 phosphate salt at about 22° C. in a variety of solvents, such as water, water/EtOH (1:1), acetonitrile/BE, methanol/BE, ethanol, acetone/heptanes, IPA, MEK/hetpanes, MIBK/heptanes, DCM/heptanes, THF, 2-MeTHF, EtOAc/heptanes, IPAc/heptanes, MTBE, and toluene. Eventually, almost all tested Compound 1 phosphate salt samples crystallized in solvate form. Only in water did Compound 1 phosphate salt remain in amorphous solid form. After drying at about 75° C. to 100° C. under vacuum, most solvate forms of Compound 1 phosphate salt became crystalline Form I, with the exception of the MeOH, acetonitrile and toluene solvates. The MeOH solvate form of Compound 1 phosphate salt only partially changed to crystalline Form I; the acetonitrile and toluene solvate forms remained unchanged.

The XRPD pattern of Compound 1 phosphate salt, crystalline Form I is shown in FIG. 20. A list of 2-theta peaks is provided in Table 6 below.

TABLE 6

| No. | Pos. [°2Th.] | Rel. Int. [%] |
|---|---|---|
| 1 | 6.8 | 23 |
| 2 | 11.8 | 26 |
| 3 | 12.2 | 11 |
| 4 | 13.4 | 30 |
| 5 | 14.7 | 4 |
| 6 | 16.1 | 13 |
| 7 | 17.9 | 100 |
| 8 | 19.4 | 8 |
| 9 | 21.2 | 16 |
| 10 | 22.0 | 16 |
| 11 | 23.7 | 10 |
| 12 | 24.4 | 9 |
| 13 | 25.3 | 10 |
| 14 | 26.6 | 13 |
| 15 | 29.1 | 5 |
| 16 | 30.2 | 6 |

The TGA thermogram of Compound 1 phosphate salt, crystalline Form I is shown in FIG. 21. A weight loss of about 1.7% from 25° C. to 100° C. was observed.

The DSC thermogram of Compound 1 phosphate salt, crystalline Form I is shown in FIG. 22, exhibiting a melting onset of about 137° C.

The DVS sorption isotherm of Compound 1 phosphate salt, crystalline Form I is shown in FIG. 23. Crystalline Form I was moderately hygroscopic, as indicated by about 2.7% water uptake over the entire range of 0% to 90% RH at 25° C.

### Example 3. (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl(1-(difluoromethyl)cyclopropyl)carbamate Maleate Salt, Crystalline Form I

Compound 1 maleate salt, crystalline Form I was first prepared as follows: A solution of 200 mg of Compound 1 free base, 1 eq. maleic acid, and 100 μL acetone was stirred at about 22° C. Following onset of crystallization and formation of a thick slurry after a few minutes of stirring, the solution was diluted with 200 μL of acetone and 150 μL of heptanes. The sample was then filtered and XRPD analysis was performed in wet form and after drying at 50° C. under vacuum. Both wet and dry solids were found to produce essentially the same XRPD pattern. Proton NMR analysis of the sample showed about 1 eq. maleic acid (data not shown).

A polymorph screening was conducted by stirring Compound 1 melaete salt, crystalline Form I in a variety of solvents, such as water, water/EtOH (1:1), acetonitrile, methanol, ethanol, acetone, IPA, MEK, MIBK, DCM, THF, 2-MeTHF, EtOAc, IPAc, MTBE, and toluene at about 22° C. Form I solids remained undissolved in each of the tested solvents. It is therefore believed that the tested solvents can be used as crystallization solvents for Compound 1 maleate salt.

The XRPD pattern of Compound 1 maleate salt, crystalline Form I is shown in FIG. 24. A list of 2-theta peaks is provided in Table 7 below.

TABLE 7

| No. | Pos. [°2Th.] | Rel. Int. [%] |
|---|---|---|
| 1 | 6.0 | 29 |
| 2 | 9.4 | 8 |
| 3 | 10.2 | 4 |
| 4 | 11.9 | 13 |
| 5 | 13.0 | 12 |
| 6 | 13.3 | 40 |
| 7 | 14.4 | 44 |
| 8 | 15.2 | 9 |
| 9 | 15.7 | 23 |
| 10 | 16.1 | 9 |
| 11 | 17.2 | 51 |
| 12 | 17.4 | 42 |
| 13 | 18.2 | 63 |
| 14 | 18.6 | 19 |
| 15 | 19.2 | 96 |
| 16 | 19.6 | 65 |
| 17 | 20.2 | 35 |
| 18 | 20.9 | 19 |
| 19 | 21.9 | 22 |
| 20 | 22.4 | 12 |
| 21 | 22.8 | 42 |
| 22 | 23.1 | 20 |
| 23 | 24.0 | 15 |
| 24 | 24.6 | 66 |
| 25 | 25.1 | 25 |
| 26 | 25.6 | 17 |
| 27 | 26.5 | 100 |
| 28 | 26.8 | 43 |
| 29 | 27.1 | 13 |
| 30 | 28.0 | 10 |
| 31 | 28.5 | 12 |
| 32 | 29.0 | 15 |
| 33 | 29.6 | 16 |
| 34 | 30.3 | 32 |
| 35 | 31.1 | 12 |
| 36 | 31.4 | 11 |
| 37 | 32.4 | 54 |
| 38 | 33.9 | 28 |
| 39 | 34.5 | 5 |
| 40 | 35.3 | 8 |
| 41 | 35.8 | 4 |
| 42 | 36.9 | 10 |
| 43 | 38.1 | 36 |
| 44 | 39.4 | 5 |

Single crystals of Compound 1 maleate salt, crystalline Form I were grown as follows: 100 mg of Compound 1 maleate salt was dissolved in 1 mL IPAc by heating to about 50° C. The solution was cooled to about 22° C. and was seeded with a small amount of Compound I maleate salt, crystalline Form I. After one day of growth SCXRD analysis was performed. FIG. 25 shows the SCXRD structure of Compound 1 maleate salt, crystalline Form I and Table 8 provides observed crystal lattice parameters. A comparison of H—C (1.879 Å) and H—N (0.862 Å) distances in a Form 1 crystal indicated that the maleic acid proton had transferred to the iminohydantoin nitrogen of Compound 1. As such Compound 1 maleate, crystalline Form 1 is a salt.

TABLE 8

| Temperature (K) | 150 | |
|---|---|---|
| Crystal system | Monoclinic | |
| Space group | I2 | |
| Unit cell parameters | a = 15.57340(12) Å | α = 90° |
| | b = 10.23151(7) Å | β = 106.2880(8)° |
| | c = 27.7130(2) Å | γ = 90° |

| | |
|---|---|
| Calculated density (g/cm³) | 1.453 |

The TGA thermogram of Compound 1 maleate salt, crystalline Form I is shown in FIG. 26. A weight loss of about 0.4% from 25° C. to 125° C. was observed, indicating that crystalline Form I is an unsolvated form of Compound 1 maleate salt.

The DSC thermogram of Compound 1 maleate salt, crystalline Form I is shown in FIG. 27, indicating an endothermic event at about 209° C.

The DVS sorption isotherm of Compound I maleate salt, crystalline Form I is shown in FIG. 28. Crystalline Form I was found to be slightly hydroscopic, as indicated by about 0.7% water uptake over the entire range of 0% to 90% RH at 25° C.

### Example 4. (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl(1-(difluoromethyl)cyclopropyl)carbamate Succinate Salt, Crystalline Form I

Compound 1 succinate salt, crystalline Form I was first prepared as follows: A solution of 200 mg of Compound 1 free base, 1 eq. succinic acid, and 200 μL IPA was stirred at at about 22° C. Crystallization occurred after stirring for 1 day. The sample was filtered and solids were analyzed by XRPD in wet form and after drying at 50° C. under vacuum. Both the wet and dry forms of Compound 1 succinate salt produced essentially the same XRPD pattern.

A polymorph screen of Compound 1 succinate salt was conducted as follows: Compound 1 succinate salt, crystalline Form I solids were charged separately into 16 individual solvents to form slurries, which were stirred at about 22° C. Sample analysis occurred after stirring for 1 week. Wet samples typically remained as Compound 1 succinate salt, crystalline Form I, except from acetone/heptanes and THF/heptanes. Solids obtained from acetone/heptanes and THF/heptanes were identified as the respective solvates of Compound 1 hemisuccinate salt (see also Example 6).

The XRPD pattern of Compound 1 succinate salt, crystalline Form I is shown in FIG. 29. A list of 2-theta peaks is provided in Table 9 below.

TABLE 9

| No. | Pos. [°2Th.] | Rel. Int. [%] |
|---|---|---|
| 1 | 6.0 | 47 |
| 2 | 9.3 | 4 |
| 3 | 10.3 | 1 |
| 4 | 11.9 | 13 |
| 5 | 13.3 | 26 |
| 6 | 14.3 | 38 |
| 7 | 15.1 | 9 |
| 8 | 15.6 | 13 |
| 9 | 16.0 | 6 |
| 10 | 17.1 | 43 |
| 11 | 17.4 | 21 |
| 12 | 18.3 | 39 |
| 13 | 18.6 | 11 |
| 14 | 19.2 | 75 |
| 15 | 19.6 | 47 |
| 16 | 20.2 | 38 |
| 17 | 20.9 | 23 |
| 18 | 21.9 | 13 |
| 19 | 22.7 | 44 |
| 20 | 23.1 | 17 |
| 21 | 24.1 | 11 |
| 22 | 24.6 | 65 |
| 23 | 25.1 | 25 |
| 24 | 25.7 | 16 |
| 25 | 26.4 | 100 |
| 26 | 26.8 | 27 |
| 27 | 28.0 | 5 |
| 28 | 28.5 | 6 |
| 29 | 29.0 | 11 |
| 30 | 29.6 | 23 |
| 31 | 30.4 | 17 |
| 32 | 31.6 | 6 |
| 33 | 32.3 | 58 |
| 34 | 33.5 | 8 |
| 35 | 33.9 | 17 |
| 36 | 34.5 | 6 |
| 37 | 35.2 | 6 |
| 38 | 35.8 | 7 |
| 39 | 36.8 | 9 |
| 40 | 38.0 | 42 |
| 41 | 38.3 | 11 |
| 42 | 38.7 | 4 |

Single crystals of Compound 1 succinate salt, crystalline Form I were grown as follows: 100 mg of Compound 1 succinate salt was dissolved in a 0.5 mL mixture of IPA/water (1:1 volume ratio) by heating to about 60° C. The sample was cooled to about 22° C. and seeded with Compound 1 succinate salt, crystalline Form I. After 12 days of growth SCXRD analysis was performed on the sample. FIG. 30 shows the SCXRD structure of Compound 1 succinate salt, crystalline Form I, and Table 10 provides observed crystal lattice parameters. A comparison of H—C (1.897 Å) and H—N (0.824 Å) distances in a Form 1 crystal indicated that the succinic acid proton had transferred to the iminohydantoin nitrogen of Compound 1. As such Compound 1 succinate, crystalline Form 1 is a salt.

TABLE 10

| Temperature (K) | 150 | |
|---|---|---|
| Crystal system | Monoclinic | |
| Space group | C2 | |
| Unit cell parameters | a = 27.6596(3) Å | α = 90° |
| | b = 10.22294(8) Å | β = 106.2803(10)° |
| | c = 15.57453(14) Å | γ = 90° |

| | |
|---|---|
| Calculated density (g/cm³) | 1.460 |

The TGA thermogram of Compound 1 succinate salt, crystalline Form I is shown in FIG. 31. A weight loss of about 0.5% from 25° C. to 125° C. was observed, indicating that crystalline Form I is an unsolvated form of Compound 1 succinate salt.

The DSC thermogram of Compound 1 succinate salt, crystalline Form I is shown in FIG. 27. The melting onset was observed at about 154° C.

The DVS option isotherm of Compound 1 succinate salt, crystalline Form I is shown in FIG. 33. Crystalline Form I was found to be slightly hydroscopic, as indicated by about 0.7% water uptake over the entire range of 10% to 90% RH at 25° C.

### Example 5. (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl (1-(difluoromethyl)cyclopropyl)carbamate Hemisuccinate Salt, Crystalline Form I

Compound 1 hemisuccinate salt, crystalline Form I was first obtained in a polymorph screening of Compound 1 succinate salt, as described in Example 5. In short, Compound 1 succinate salt, crystalline Form I solids were charged into a variety of solvents to form slurries, which were stirred at about 22° C. The samples were analyzed after stirring for 1 week. Most wet samples remained as Compound 1 succinate salt, crystal Form I, whereas samples from acetone/heptanes and THF/heptanes, were identified as solvate forms of Compound 1 hemisuccinate salt. After drying at 70° C. under vacuum, acetone/heptane solvates and THF/heptane solvates desolvated to become Compound 1 hemisuccinate salt, crystalline Form I.

Compound 1 hemisuccinate salt, crystalline Form I was also obtained by stirring an IPA solution of Compound 1 free base and 0.5 eq. succinic acid 3 days at about 22° C. Compound 1 monosuccinate salt crystallized under these conditions. The slurry was dissolved in acetone and dried to an amorphous solid, which was stirred in a variety of solvents at about 22° C. Results are shown in Table 11. Solids from acetone/heptanes, DCM, toluene/heptanes, 1,4-dioxane/heptane, and diisopropyl ether were confirmed as solvates of Compound 1 hemisuccinate salt. Solids from DCM, toluene/heptanes and IPE were dried at 50° C. and conversion to Compound 1 hemisuccinate salt, crystalline Form 1 was shown by XRPD. Proton NMR demonstrated that Compound 1 hemisuccinate salt, crystalline Form I prepared from IPE contained about a half equivalent of succinic acid (data not shown).

TABLE 11

| Solvent | Wet form | Dry form |
|---|---|---|
| Water | Amorphous | NA |
| Water/EtOH (1:1) | Amorphous | NA |
| Acetone/heptanes | Acetone solvate of hemisuccinate | NA |
| MIBK/heptanes | Compound 1 succinate salt, | NA |
| | crystalline Form I | |
| DCM | DCM solvate of hemisuccinate | Hemisuccinate |
| | | Form I |
| 2-MeTHF/heptanes | Compound 1 succinate salt, | NA |
| | crystalline Form I | |
| IPAc/heptanes | Compound 1 succinate salt, | NA |
| | crystalline Form I | |
| MTBE/heptanes | Compound 1 succinate salt, | NA |
| | crystalline Form I | |
| Toluene/heptanes | Toluene solvate of hemisuccinate | Hemisuccinate |
| | | Form I |
| Butyronitrile/heptanes | Compound 1 succinate salt, | NA |
| | crystalline Form I | |
| 2-BuOH/heptanes | Compound 1 succinate salt, | NA |
| | crystalline Form I | |
| Cyclohexanone/heptanes | Compound 1 succinate salt, | NA |
| | crystalline Form I | |
| 1,4-Dioxane/heptanes | Dioxane solvate of hemisuccinate | NA |
| diisopropyl ether | IPE solvate of hemisuccinate | Hemisuccinate |
| | | Form I |
| Propylene glycol | Compound 1 succinate salt, | NA |
| | crystalline Form I | |

NA refers to no data available.

The XRPD pattern of Compound 1 hemisuccinate salt, crystalline Form I is shown in FIG. 34. A list of 2-theta peaks is provided in Table 12 below.

TABLE 12

| No. | Pos. [°2Th.] | Rel. Int. [%] |
|---|---|---|
| 1 | 6.5 | 68 |
| 2 | 8.7 | 23 |
| 3 | 9.6 | 18 |
| 4 | 11.2 | 13 |
| 5 | 11.8 | 18 |
| 6 | 13.4 | 17 |
| 7 | 14.7 | 33 |
| 8 | 15.1 | 34 |
| 9 | 16.1 | 14 |
| 10 | 17.4 | 95 |
| 11 | 17.9 | 100 |
| 12 | 18.5 | 62 |
| 13 | 19.1 | 61 |
| 14 | 19.8 | 74 |
| 15 | 21.1 | 40 |
| 16 | 26.8 | 20 |
| 17 | 29.6 | 11 |

The TGA thermogram of Compound 1 hemisuccinate salt, crystalline Form I is shown in FIG. 35. A weight loss of about 1% from 25° C. to 110° C. was observed.

The DSC thermogram of Compound 1 hemisuccinate salt, crystalline Form I is shown in FIG. 36. An endothermal event was observed at about 99° C.

### Example 6. Form Comparison

One measure of the crystallinity of the Compound 1 salt crystalline forms provided herein is the heat of fusion. While HC1 is a stronger acid than, e.g., maleic acid or succinic acid, lower heats of fusion were observed for all three crystalline forms of Compound 1 hydrochloride salt compared to Compound 1 maleate and succinate salts. Also crystalline forms of Compound 1 maleate and succinate salts provided the lowest hygroscopicity observed for any Compound 1 salt form provided herein. A comparison of the properties of Compound 1 salt crystalline forms is provided in Table 13 below.

TABLE 13

| | | Melting Temperature | Heat of Fusion | DVS |
|---|---|---|---|---|
| Compound 1 Salt | Form | (° C.) | (J/g) | (%) |
| HCl salt | Form I | 140 | 18 | 1.8 |
| | Form II | 141 | 14 | 3 |
| | Form III | 145 | 23 | 1.4 |
| Phosphate | Form I | 137 | 16 | 2.7 |
| Maleate | Form I | 209 | 128 | 0.8 |
| Succinate | Form I | 154 | 70 | 0.7* |
| Hemisuccinate | Form I | 99 | 21 | NA |

*Measured at 10% to 90% RH. Others were measured at 0% to 90% RH

### Example 7. Polymorph Screening of benzyl ((R,E)-1-((S)-1-(4-chloro-3-(1-(difluoromethyl)-1H-1,2,4-triazol-5-yl)phenyl)-2-(((1-(difluoromethyl)cyclopropyl)carbamoyl)oxy)ethyl)-4-(4-(2-cyclopropyl-2H-1,2,3-triazol-4-yl)phenyl)-5-oxo-4-(3,3,3-trifluoro-2,2-dimethylpropyl)imidazolidin-2-ylidene)carbamate

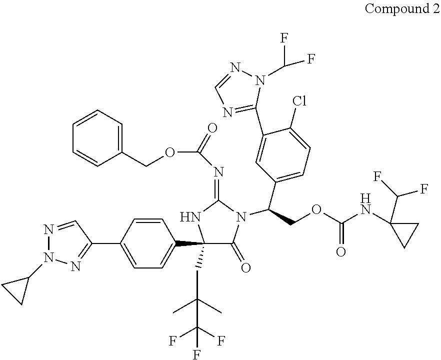 [SMILES: CC(C)(C[C@]1(c2ccc(-c3cnn(C4CC4)n3)cc2)N/C(=N\C(=O)OCc2ccccc2)N([C@H](COC(=O)NC2(C(F)F)CC2)c2ccc(Cl)c(-c3ncnn3C(F)F)c2)C1=O)C(F)(F)F]

A polymorph screening was carried out by stirring Compound 2 at about 22° C. in 18 solvents/mixtures: IPA, 2-BuOH, MIBK/heptanes, IPAc/heptanes, CPME/heptanes, Toluene, IPE, MeCN/BE, acetone/heptanes, MEK/hetpanes, DCM/heptanes, 2-MeTHF/hetpanes, MTBE/hetpanes, water, MeOH/BE, EtOH/hetpanes, THF/hetpanes, and EtOAc/heptanes. Crystalline solvate forms of Compound 2 were obtained from MIBK/heptanes and MEK/heptanes. The XRPD profile of the MEK solvate form of Compound 2 is shown in FIG. 37. The XRPD profile of the MIBK solvate form of Compound 2 is shown in FIG. 38.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure.

1. A crystalline form of (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl (1-(difluoromethyl)cyclopropyl)carbamate hydrochloride salt, which is selected from crystalline Form I, crystalline Form II, and crystalline Form III.

2.-3. (canceled)

4. The crystalline form of claim 1, wherein the crystalline Form I is characterized by an XRPD pattern comprising three peaks, in terms of 2-theta±0.2°, selected from 6.2°, 10.4°, 12.6°, 13.7°, 16.3°, 17.8°, 21.7°, 22.2°, and 26.8°.

5.-11. (canceled)

12. The crystalline form of claim 1, wherein the crystalline Form I is characterized by an XRPD pattern substantially as shown in FIG. 3.

13. The crystalline form claim 1, wherein the crystalline Form I is characterized by a DSC thermogram having a melting onset of about 140° C.

14. The crystalline form of claim 1, wherein the crystalline Form I is characterized by a DSC thermogram substantially as shown in FIG. 5.

15. The crystalline form of claim 1, wherein the crystalline Form II is characterized by an XRPD pattern comprising three peaks, in terms of 2-theta±0.2°, selected from 6.5°, 7.2°, 8.5°, 11.3°, 13.4°, 14.3°, 15.7°, 17.0°, and 17.7°.

16.-22. (canceled)

23. The crystalline form of claim 1, wherein the crystalline Form II is characterized by an XRPD pattern substantially as shown in FIG. 8.

24. The crystalline form of claim 1, wherein the crystalline Form II is characterized by a DSC thermogram having a melting onset of about 141° C.

25. The crystalline form of claim 1, wherein the crystalline Form II is characterized by a DSC thermogram substantially as shown in FIG. 10.

26. The crystalline form of claim 1, wherein the crystalline Form III is characterized by an XRPD pattern comprising three peaks, in terms of 2-theta±0.2°, selected from 6.3°, 6.6°, 10.9°, 13.7°, 15.3°, 17.3°, 18.5°, 20.0°, and 25.1°.

27.-34. (canceled)

35. The crystalline form of claim 1, wherein the crystalline Form III is characterized by an XRPD pattern substantially as shown in FIG. 14.

36. The crystalline form of claim 1, wherein the crystalline Form III is characterized by a DSC thermogram having a melting onset of about 145° C.

37. The crystalline form of claim 1, wherein the crystalline Form III is characterized by a DSC thermogram substantially as shown in FIG. 16.

38. A crystalline form of (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3 ,3 ,3-trifluoro-2,2-dimethylpropyl)imidazolidin-1-yl)ethyl (1-(difluoromethyl)cyclopropyl)carbamate phosphate salt, which is crystalline Form I.

39.-40. (canceled)

41. The crystalline form of claim 38, wherein the crystalline Form I is characterized by an XRPD pattern comprising three peaks, in terms of 2-theta±0.2°, selected from 6.8°, 11.8°, 12.2°, 13.4°, 16.1°, 17.9°, 21.2°, 22.0°, and 25.3°.

42.-48. (canceled)

49. The crystalline form of claim 38, wherein the crystalline Form I is characterized by an XRPD pattern substantially as shown in FIG. 20.

50. The crystalline form of claim 38, wherein the crystalline Form I is characterized by a DSC thermogram having a melting onset of about 137° C.

51. The crystalline form of claim 38, wherein the crystalline Form I is characterized by a DSC thermogram substantially as shown in FIG. 22.

52. A crystalline form of (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl (1-(difluoromethyl)cyclopropyl)carbamate maleate salt, which is crystalline Form I.

53.-54. (canceled)

55. The crystalline form of claim 52, wherein the crystalline Form I is characterized by an XRPD pattern comprising three peaks, in terms of 2-theta±0.2°, selected from 6.0°, 13.3°, 14.4°, 17.2°, 18.2°, 19.2°, 19.6°, 24.6°, and 26.5°.

56.-62. (canceled)

63. The crystalline form of claim 52, wherein the crystalline Form I is characterized by an XRPD pattern pattern substantially as shown in FIG. 24.

64. The crystalline form of claim 52, wherein the crystalline Form I is characterized by a DSC thermogram having a melting onset of about 209° C.

65. The crystalline form of claim 52, wherein the crystalline Form I is characterized by a DSC thermogram substantially as shown in FIG. 27.

66. A crystalline form of (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3 ,3 ,3-trifluoro-2,2-dimethylpropyl)imidazolidin-1-yl)ethyl (1-(difluoromethyl)cyclopropyl)carbamate succinate salt, which is crystalline Form I.

67.-68. (canceled)

69. The crystalline form of claim 66, wherein the crystalline Form I is characterized by an XRPD pattern comprising three peaks, in terms of 2-theta±0.2°, selected from 6.0°, 13.3°, 14.3°, 17.1°, 18.3°, 19.2°, 19.6°, 24.6°, and 26.4°.

70.-76. (canceled)

77. The crystalline form of claim 66, wherein the crystalline Form I is characterized by an XRPD pattern substantially as shown in FIG. 29.

78. The crystalline form of claim 66, wherein the crystalline Form I is characterized by a DSC thermogram having a melting onset of about 154° C.

79. The crystalline form of claim 66, wherein the crystalline Form I is characterized by a DSC thermogram substantially as shown in FIG. 32.

80. A crystalline form of (S)-2-(4-chloro-3-(1-(difluoromethyl)-1H-1,2,4-triazol-5-yl)phenyl)-2-((R)-4-(4-(2-cyclopropyl-2H-1,2,3-triazol-4-yl)phenyl)-2-imino-5-oxo-4-(3,3,3-trifluoro-2,2-dimethylpropyl)imidazolidin-1-yl)ethyl (1-(difluoromethyl)cyclopropyl)carbamate hemisuccinate salt, which is crystalline Form I.

81.-82. (canceled)

83. The crystalline form of claim 80, wherein the crystalline Form I is characterized by an XRPD pattern comprising three peaks, in terms of 2-theta±0.2°, selected from 6.5°, 8.7°, 15.1°, 17.4°, 17.9°, 18.5°, 19.1°, 19.8°, and 21.1°.

84.-90. (canceled)

91. The crystalline form of claim 80, wherein the crystalline Form I is characterized by an XRPD pattern substantially as shown in FIG. 34.

92. A pharmaceutical composition comprising the crystalline form of claim 1, and at least one pharmaceutically acceptable excipient.

93. A method of treating or preventing a human immunodeficiency virus (HIV) infection comprising administering a therapeutically effective amount of the crystalline form of claim 1 to a subject in need thereof.

94. The method of claim 93, wherein the method comprises administering the crystalline form in combination with one, two, three, or four additional therapeutic agents.

95. The method of claim 94, wherein the additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors, cell therapies, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, CD4 modulators, CD4 antagonists, HIV ribonuclease H inhibitors, retrocyclin modulators, CDK-9 inhibitors, CCR5 chemokine antagonists, CCR5 gene modulators, dendritic ICAM-3 grabbing nonintegrin 1 inhibitors, HIV GAG protein inhibitors, HIV POL protein inhibitors, hyaluronidase inhibitors, Nef antagonists, Nef inhibitors, Protease-activated receptor-1 antagonists, TNF alpha ligand inhibitors, PDE4 inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, long acting HIV regimens, contraceptives, or any combinations thereof.

96. The method of claim 94, wherein the additional therapeutic agents are selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, or any combinations thereof.

97. The method of claim 94, wherein the additional therapeutic agents are selected from the group consisting of 4′-ethynyl-2-fluoro-2′-deoxyadenosine, bictegravir or a pharmaceutically acceptable salt thereof, abacavir sulfate, tenofovir, tenofovir disoproxil, tenofovir disoproxil fumarate, tenofovir disoproxil hemifumarate, tenofovir alafenamide, and tenofovir alafenamide hemifumarate.

98. The method of claim 94, wherein the additional therapeutic agents are selected from the group consisting of 4′-ethynyl-2-fluoro-2′-deoxyadenosine, bictegravir or a pharmaceutically acceptable salt thereof, tenofovir alafenamide, tenofovir alafenamide fumarate and tenofovir alafenamide hemifumarate.

99.-112. (canceled)
